(19) United States
(12) Patent Application Publication
(10) Pub. No.: US 2004/0210107 A1
(43) Pub. Date: Oct. 21, 2004

(54) Endoscope system for fluorescent observation

(75) Inventors: Nobuhiro Tani, Tokyo (JP); Hideo Sugimoto, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 10/811,974

(22) Filed: Mar. 30, 2004

(30) Foreign Application Priority Data
Mar. 31, 2003 (JP) 2003-094996
Mar. 31, 2003 (JP) 2003-094997
Mar. 31, 2003 (JP) 2003-094998

(51) Int. Cl. A61B 1/04

(52) U.S. Cl. 600/109; 600/160

(57) ABSTRACT

There is provided an endoscope system, which is provided with an endoscope that has an image pick-up device having a plurality of cells for accumulating charge in response to an image formed on the plurality of cells, a light source that is capable of emitting excitation light, and a controlling system that controls the image pick-up device so that charges accumulated in two or more of the plurality of cells are added together when the excitation light is used.

### BACKGROUND OF THE INVENTION

The present invention relates to an endoscope system which is capable of observing a fluorescence image generated by emission of excitation light to an object.

Endoscope systems of this type have come into widespread use. Japanese Patent Provisional Publication No. HEI 8-557 discloses one of such endoscope systems. The endoscope system disclosed in this publication includes a light source unit which can illuminate an object (e.g., human tissue) with excitation light as well as normal light, and a fiberscope (endoscope).

The fiberscope includes an objective lens which forms an image, a fiber bundle, and an eyepiece. The image formed by the objective lens is transferred through the fiber bundle and the eyepiece to an external image intensifier provided in an adapter system connected to the fiberscope. The image intensifier enhances the image transferred from the fiber scope so that image data having sufficient intensity for observation can be obtained by an image pickup device such as a CCD (charge coupled device).

Japanese Patent Provisional Publication No. HEI 9-70384 also discloses an endoscope system which uses an image intensifier to enhance an image formed by an objective lens in an endoscope.

However, the endoscope systems disclosed in the above mentioned publications require use of the expensive image intensifier to obtain the image for fluorescent observation (i.e., a fluorescence image), which increases cost of the endoscope system.

Further, in the endoscope system disclosed in HEI 8-557, the image formed by the objective lens is transferred to the image intensifier via the fiber bundle provided in the fiberscope and a fiber bundle provided in the adapter system. Such configuration of the endoscope system requires that the length of the fiber bundle in the adapter system is as short as possible to decrease loss of intensity of the image in the fiber bundle of the adapter system. If the length of the fiber bundle of the adapter system is set short to avoid such loss of image quality, handling of the fiberscope may be limited.

### SUMMARY OF THE INVENTION

The present invention is advantageous in that it provides an endoscope system which is capable of amplifying an image formed by fluorescence emitted from an object.

According to an aspect of the invention, there is provided an endoscope system, which is provided with an endoscope that has an image pick-up device having a plurality of cells for accumulating charge in response to an image formed on the plurality of cells, a light source that is capable of emitting excitation light, and a controlling system that controls the image pick-up device so that charges accumulated in two or more of the plurality of cells are added together when the excitation light is used.

With this configuration, an image for fluorescent observation can be amplified without using an expensive image intensifier.

Optionally, the image pick-up device may include a charge coupled device.

In a particular case, the charge coupled device may have a frame-transfer architecture.

In a particular case, the charge coupled device may have an interline architecture.

Optionally, the controlling system may include: a driver circuit that outputs timing signals to the charge coupled device, the charge coupled device outputting charge accumulated in the plurality of cells in accordance with the timing signals; a detection amplifier that accumulates charge outputted by the charge coupled device and outputs voltage levels in accordance with the charge accumulated therein; and a correlated double sampling circuit that samples the voltage levels outputted by the detection amplifier and outputs a sampled signal.

Still optionally, the plurality of cells in the charge coupled device may be arranged in a matrix, and the charge coupled device may include a horizontal transferring charge coupled device connected to the plurality of cells in the charge coupled device, the horizontal transferring charge coupled device outputting the charge to the detection amplifier.

Still optionally, charge accumulated in each of the plurality of cells arranged in the matrix may be transferred vertically a plurality of times toward the horizontal transferring charge coupled device so that charge of at least two cells aligned vertically in the plurality of cells is accumulated in each cell of the horizontal transferring charge coupled device.

Still optionally, charge accumulated in each of the cells in the horizontal transferring charge coupled device may be transferred horizontally a plurality of times so that charge of at least two cells in the horizontal transferring charge coupled device is accumulated in the detection amplifier.

Still optionally, the driving circuit may transmit reset pulses to the detection amplifier to discharge the charge accumulated in the detection amplifier, and may transmit sampling signals to the correlated double sampling circuit to sample the voltage levels outputted by the detection amplifier. In this case, the driving circuit may control timing of the reset pulses and the sampling signals so that charge of the at least two cells aligned vertically in the plurality of cells is accumulated in each cell of the horizontal transferring charge coupled device.

In a particular case, the endoscope system may include an image processing system that generates an image for fluorescent observation so that an intensity of one pixel of the image corresponds to the added charges of the two or more of the plurality of cells.

In a particular case, the image pick-up device may include a color CCD having color filter covering the plurality of cells. In this case, the controlling system may control the color CCD so that a number of cells whose charges are added together becomes an integral multiple of a number of filter elements of the color filter to be used for generating a pixel of an image for fluorescent observation.

Optionally, the endoscope system may include a memory that stores information concerning a repetition pattern of the filter elements, and the controlling system may obtain the number of filter elements to be used for generating the pixel of the image for fluorescent observation based on the information stored in the memory.

In a particular case, the image pick-up device may include a color CCD having color filter covering the plurality of cells, and the controlling system may control the color CCD so that a number of cells whose charges are added together becomes an integral multiple of a number of filter elements of the color filter to be used for generating a pixel of an image for fluorescent observation. In this case, the control system may control the color CCD so that a combination of the filter elements, to which the two or more cells added together correspond, is changed between an odd field and an even field.

Optionally, the endoscope system may include an image processing system that generates an interlace scan image signal so that an intensity of one pixel of the interlace scan image signal corresponds to the added charges of the two or more of the plurality of cells.

### BRIEF DESCRIPTION OF THE ACCOMPANYING DRAWINGS

FIG. 1 is a block diagram of an endoscope system according to a first embodiment of the invention;

FIG. 2 schematically shows a configuration of a CCD unit in the endoscope system in FIG. 1;

FIG. 3 is a timing chart showing switching of emission of light, and timing of a select signal and timing signals;

FIG. 4 is a timing chart showing timing of a φV signal, a φH signal and a reset pulse during a period of non-emission period in FIG. 3;

FIG. 5 is a timing chart showing the timing of the φH signal and the reset pulse during a period in FIG. 4;

FIG. 6 is a timing chart showing the φV signal, the φH signal and the reset pulse during a period in the non-emission period following UV light emission period;

FIG. 7 is a timing chart showing the φV signal, the φH signal and the reset pulse during a period in FIG. 6;

FIG. 8 shows a block diagram of an endoscope system according to a second embodiment of the invention;

FIG. 9 shows a configuration of a CCD unit in the endoscope system shown in FIG. 8;

FIG. 10 shows another example of the CCD unit having an interline architecture;

FIG. 11 is a flowchart showing an image processing according to the second embodiment;

FIG. 12 is a flowchart showing an EEPROM read process performed in the image processing shown in FIG. 11;

FIG. 13 shows an example of color filter array patterns;

FIG. 14 shows another example of the color filter array pattern;

FIG. 15 shows another example of the color filter array pattern;

FIG. 16 is a flowchart showing a normal image obtaining process performed in the image processing shown in FIG. 11;

FIG. 17 is a timing chart showing timing signals outputted by a driver circuit when the driver circuit receives Px and Py from a MPU;

FIG. 18 is a timing chart showing timing within a timing period in FIG. 17 in detail;

FIG. 19 is a flowchart showing in detail a fluorescence image obtaining process performed in the flow of FIG. 11;

FIG. 20 is a timing chart showing timing signals outputted by a driver circuit when Bx and By are transmitted from a MPU to the driver circuit;

FIG. 21 is a timing chart showing in detail timing of the timing signals outputted during a time period in FIG. 20;

FIG. 22 shows a block diagram of an endoscope system according to a third embodiment of the invention;

FIG. 23 shows a configuration of a CCD unit in FIG. 22;

FIG. 24 shows another example of a CCD unit having a interline architecture;

FIG. 25 is a flowchart showing an image processing according to the third embodiment;

FIG. 26 is a flowchart showing an EEPROM read process performed in the image processing shown in FIG. 25;

FIG. 27 is a flowchart showing a normal image obtaining process performed in the image processing shown in FIG. 25;

FIG. 28 is a timing chart showing timing signals outputted by a driver circuit;

FIG. 29 is a timing chart showing timing within a timing period in FIG. 28 in detail;

FIG. 30 is a flowchart showing in detail a fluorescence image obtaining process;

FIG. 31 is a timing chart showing the timing signals outputted by the driver circuit when Bx, By and FC (FC=1) are transmitted from a MPU 601 the driver circuit;

FIG. 32 is a timing chart showing in detail the timing of the timing signals outputted during a time period FIG. 31;

FIG. 33 is a timing chart showing the timing signals outputted by the driver circuit when the Bx, By and FC (FC=0) are transmitted from the MPU to the driver circuit;

FIG. 34 is a timing chart showing in detail the timing of the timing signals outputted during a time period in FIG. 33;

FIG. 35 shows the upper left portion of a CCD on which a color filter shown in FIG. 13 is formed, schematically illustrating cells which are used to generate one field of the fluorescence image when the parameters are FC=1, Ax=2, Ay=2, Px=2 and Py=2; and

FIG. 36 shows the upper left portion of the CCD on which the color filter shown in FIG. 13 is formed, schematically illustrating cells which are used to generate one field of the fluorescence image when the parameters are FC=0, Ax=2, Ay=2, Px=2 and Py=2.

### DETAILED DESCRIPTION OF THE EMBODIMENTS

Hereinafter, embodiments according to the invention are described with reference to the accompanying drawings.

First Embodiment

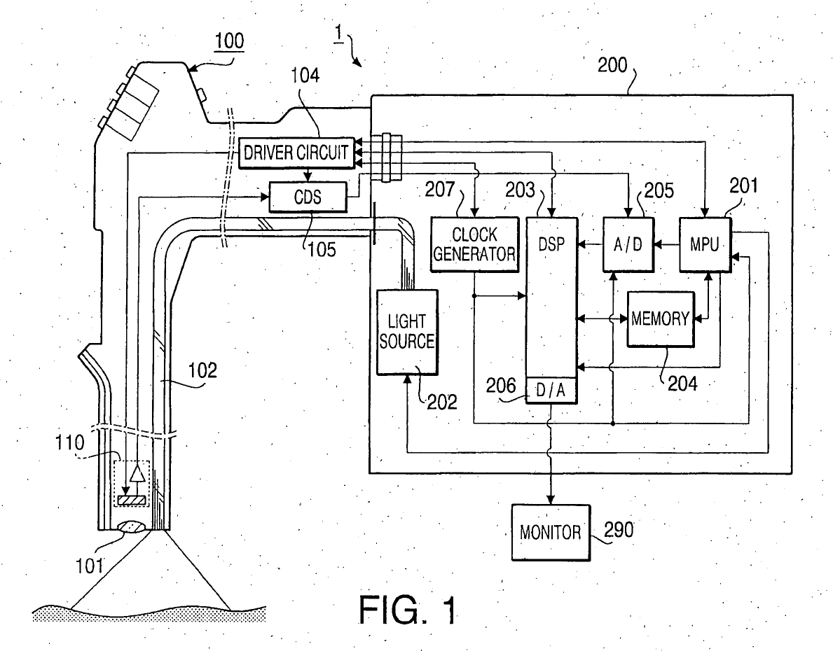

FIG. 1 is a block diagram of an endoscope system 1 according to a first embodiment of the invention. The endoscope system 1 includes an endoscope (i.e., an electronic endoscope) 100, an endoscope processor 200 and a monitor 290. The endoscope processor 200 is configured to selectively emit light for normal observation and excitation light for fluorescent observation. An image captured by the endoscope 100 is transmitted to the endoscope processor 200 to process the image and to generate a video signal. The video signal is then transmitted to the monitor 290 on which the image of an object (e.g., human tissue) is displayed.

As shown in FIG. 1, the endoscope 100 includes an objective lens 101, a CCD unit 110 on which an image is formed by the objective lens 101, a fiber bundle (i.e., a light guide) 102, a driver circuit 104, and a CDS (Correlated Double Sampling) circuit 105. The endoscope processor 200 includes a MPU (microprocessor unit) 201, a light source 202, a DSP (digital signal processor) 203, a memory 204, an A/D converter 205, a D/A converter 206 and a clock generator 207.

A proximal end portion of the fiber bundle 102 is connected optically to the light source 202. A distal end of the fiber bundle 102 is positioned at a distal end of an insertion tube of the endoscope 100. Light emitted from the light source 202 is transmitted through the fiber bundle 102 to illuminate the object from the distal end of the fiber bundle 102. As described in detail later, the light source 202 emits sequentially red light, green light, blue light, and the excitation light to obtain color images and fluorescence images.

The objective lens 101 forms the image of the object which is illuminated by light from the light source 202 onto a light receiving surface of the CCD unit 110 driven by timing signals from the driver circuit 104.

The CCD unit 110 outputs an image signal in synchronization with the timing signals from the driver circuit 104. The image signal corresponding to one frame of the image is outputted during each of emission periods of light. During the one emission period, one of the red light, green light, blue light and excitation light is emitted.

The CDS circuit 105 receives the image signal from the CCD unit 110 and processes the image signal to generate a CDS signal having an appropriate form for the A/D converter 205. The A/D converter 205 process the CDS signal into digitized form. The digitized signal is transmitted from the A/D converter 205 to the DSP 203 in which image processing such as a γ-correction is performed on the digitized image signal to store the processed image signal as image data in the memory 204.

In the memory 204, image data respectively corresponding to red image data (captured during illumination of red light), green image data (captured during illumination of green light) and blue image data (captured during illumination of blue light), and fluorescence image data (captured during illumination of the excitation light) are stored.

Further, the DSP 203 reads out the image data from the memory 204 and combines them. Then, the combined image data is processed by the D/A converter 206 having a video encoder to generate an analog video signal (e.g., an NTSC signal). Then, the video signal is transmitted to the monitor 290.

The above mentioned processes are performed under control of the MPU 201 which executes various programs stored in, for example, a ROM embedded therein. That is, the MPU 201 controls the light source 202, the DSP 203, the memory 204, the A/D converter 205 and the D/A converter 206. For example, a sampling rate of the A/D converter 205 is determined by the MPU 201. The sampling rate determined by the MPU 201 is used by the A/D converter 205 to digitize the CDS signal at the sampling rate, and is also used by the DSP 203 to generate the image data.

The clock generator 207 generates various timing signals including a vertical synchronizing signal. The synchronizing signals are sent to the driver circuit 104, the DSP 203, and the A/D converter 205. The driver circuit 104 provided in the endoscope 100 generates timing signals based on the synchronizing signals from the clock generator 207 and sends the timing signals to the CCD unit 110 and the CDS circuit 105.

The CCD unit 110 operates in synchronization with the timing signal from the driver circuit 104 to capture the image. The CDS circuit 105 processes the image signal from the CCD unit 110 in synchronization with the timing signal from the driver circuit 104. The A/D converter 205 digitizes the CDS signal based on the timing signals from the clock generator 207 and the determined sampling rate.

The DSP 203 can calculate resolution (i.e., pixel numbers in the vertical direction and in the horizontal direction) of the image data based on the timing signals and the determined sampling rate, and converts the signal from the A/D converter 205 into the image data using the calculated resolution.

Further, based on the synchronizing signals, the MPU 201 controls the light source 202 to determine timing of emission of each of the red light, green light, blue light and excitation light. At the timing of the vertical synchronizing signal, the type of light is switched to another. Each time each of the red light, green light, blue light and excitation light is emitted, the image signal corresponding to one frame is obtained.

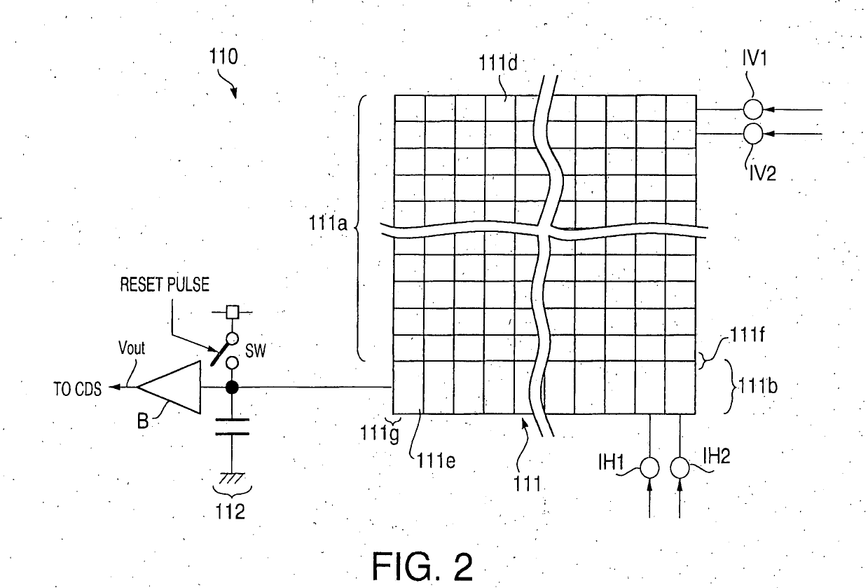

FIG. 2 schematically shows a configuration of the CCD unit 110. As shown in FIG. 2, the CCD unit 110 includes a CCD (charge coupled device) 111, a Floating Diffusion Amplifier (i.e., a charge detection amplifier) 112, and buffer B. As shown in FIG. 2, the CCD unit 110 has a frame-transfer architecture. However, the CCD unit 110 may be replaced with a CCD unit having an interline architecture.

The CCD 110 includes a charge accumulation unit 111a on which the image is formed by the objective lens 101, a horizontal transferring unit 111b, horizontal pulse input terminals IH1 and IH2, and vertical pulse input terminals IV1 and IV2. The charge accumulation unit 111a has a plurality of cells 111d arranged in a form of n by n pixel matrix.

The horizontal transferring unit 111b has a plurality of cells 111e arranged horizontally in a line and is adjacent to a side 111f of the charge accumulation unit 111a. The number of cells 111e in the horizontal transferring unit 111b is equal to the number of cells 111d arranged horizontally along the side 111f.

The cells 111e in the horizontal transferring unit 111b are respectively connected to the cells 111d arranged along the side 111f. One of the cells 111e located at one end (i.e., the leftward end in FIG. 2) of the horizontal transferring unit 111b is connected to the FDA 112. The horizontal pulse input terminals IH1 and IH2, and the vertical pulse input terminals IV1 and IV2 are connected to the driver circuit 104.

While the optical image is formed by the objective lens 101 on the cells 111d, charge is accumulated in each cell 111d. The amount of charge accumulated in each cell 111d depends on an accumulation period (i.e., light emission period) and a luminance of the optical image. Each time pulses are inputted to the vertical pulse input terminals IV1 and IV2, charge accumulated in each cell 111d is transferred vertically (i.e., downward in FIG. 2) to its neighboring cell toward the horizontal transferring unit 111b. Also, charges accumulated in cells 111d arranged along the side 111f are respectively transferred to the cells 111e of the horizontal transferring unit 111b.

Each time pulses are inputted to the horizontal pulse input terminals IH1 and IH2, charge accumulated in each cell 111e is transferred horizontally (i.e.. leftward in FIG. 2) to its neighboring cell 111e. Also, charge accumulated in one of cells 111e located nearest to the FDA 112 is transferred to the FDA 112.

The FDA 112 converts the charge transferred from the horizontal transferring unit 111b to a voltage level. The FDA 112 has a switch SW which is switched to on or off by control of a reset pulse from the driver circuit 104. When the reset pulse is inputted to the FDA 112, the charge accumulated therein is discharged, and thereafter next accumulation of charge starts. The voltage level generated by the FDA 112 is thus transmitted to the CDS circuit 105 through the buffer B. The buffer B is a wide-band buffer which outputs signal at low impedance. Thus, the image signal (i.e., Vout) generated by the CCD unit 110 is transmitted to the CDS circuit 105.

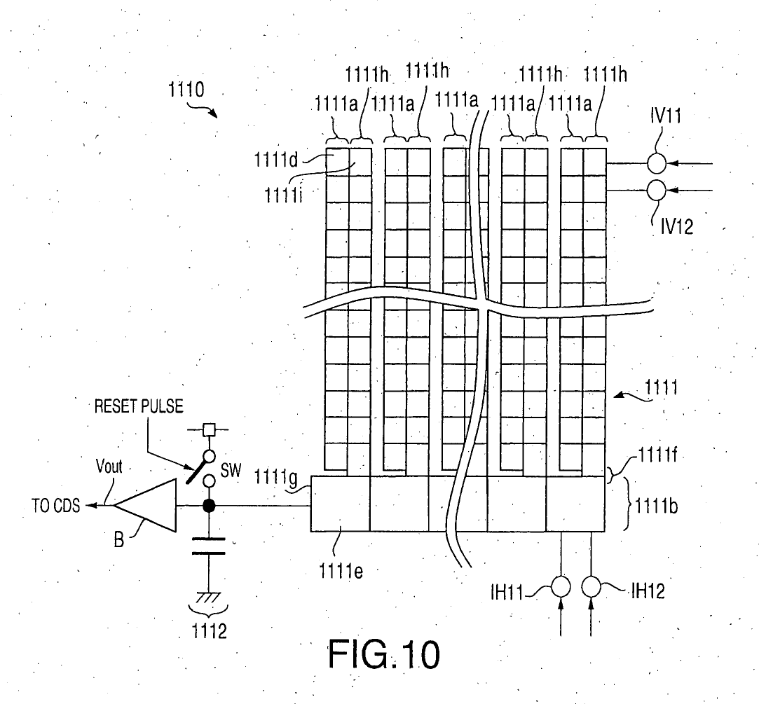

As described above, the CCD unit 110 may be replaced with the CCD unit having the interline architecture. The CCD having the interline architecture can be controlled by using a control scheme substantially the same as the above mentioned control scheme for the CCD having the frame-transfer architecture, as described in detail later in a second embodiment with reference to FIG. 10.

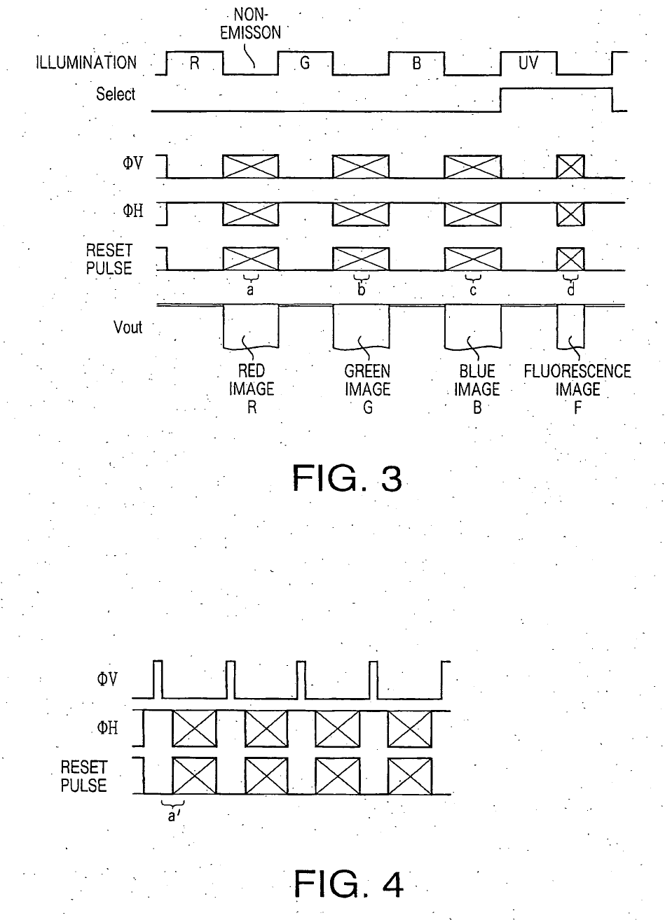

Hereafter, detailed explanations of the timing of various signals of the endoscope system 1 will be made with reference to FIGS. 3-7. FIG. 3 is a timing chart showing the switching of the emission of light, the timing of a select signal which is transmitted from the MPU 201 to the driver circuit 104, the timing of a pulse φH inputted to the horizontal pulse input terminals IH1 and IH2, and the timing of a pulse φV inputted to the vertical pulse input terminals IV1 and IV2. Further, in FIG. 3, the timing of the reset pulse, and the timing of the image signal transmitted from the buffer B to the CDS circuit 105 are indicated.

As shown in FIG. 3, a sequence of emission of red light, green light, blue light and excitation light is repeated periodically. In FIG. 3, one of such sequences is indicated for the sake of simplicity. As shown in FIG. 3, after the red light emission period, a non-emission period is provided, followed by the green light emission period, a non-emission period, a blue light emission period, a non-emission period, a UV (ultraviolet) light emission period (i.e., excitation light emission period), and a non-emission period.

The select signal is asserted during the UV light emission period and the succeeding non-emission period. The φV signal, the φH signal and the reset pulse are asserted only during the non-emission periods. Therefore, the image signal is outputted by the CCD unit 110 during the non-emission periods. That is, during the non-emission period following the red light emission, the image signal of the image formed on the charge accumulation unit 111a by the red light emission is outputted. During the non-emission period following the green light emission, the image signal of the image formed on the charge accumulation unit 111a by the green light emission is outputted. During the non-emission period following the blue light emission, the image signal of the image formed on the charge accumulation unit 111a by the blue light emission is outputted.

During the non-emission period following the UV light emission, the image signal of the image (i.e., the fluorescence image) formed on the charge accumulation unit 111a by the UV light emission is outputted.

FIG. 4 is a timing chart showing the φV signal, the φH signal and the reset pulse during a period “a” of the non-emission period indicated in FIG. 3. As shown in FIG. 4, the φV signal is asserted periodically during the non-emission period. Between two φV pulses, the φH pulses and the reset pulses are asserted. Specifically, two kinds of vertical synchronizing signals slightly shifted from each other are respectively inputted to the vertical pulse input terminals IV1 and IV2. However, the two kind of vertical synchronizing signals are represented by the φV signal in the timing charts of this embodiment for the sake of simplicity.

Each time one pulse φV is inputted to the CCD unit 110, the charge accumulated in each cell 111d is transferred by one step to its neighboring cell toward the horizontal transferring unit 111b. During the one non-emission period, pulses φV are inputted to the CCD unit 110 “n” times. It is noted that the timing chart of FIG. 4 is also applied to the timing during the non-emission periods of “b”, and “c” shown in FIG. 3 respectively following the green light emission period and the blue light emission period.

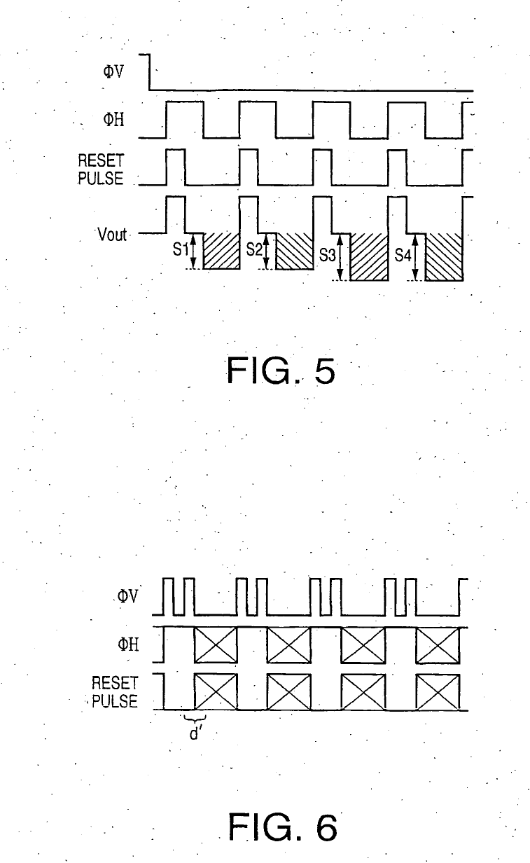

Next, detailed timing of the φH signal and the reset pulse will be described. FIG. 5 is a timing chart showing the timing of the φH signal and the reset pulse during a period “a′” indicated in FIG. 4. As shown in FIG. 5, the φH signal is periodically asserted n times while the φV signal is low. The reset pulse rises in synchronization with rising edge of the φH signal, and falls before the φH signal falls.

Following the rising edge of each pulse φH, charge is transferred to the FDA 112, and the voltage level (S1,S2,S3 and S4) corresponding to the charge accumulated in the FDA 112 is outputted from the FDA 112 until the next reset pulse is inputted to the FDA 112. Therefore, charges accumulated in the cells 111e of the horizontal transferring unit 111b are outputted in synchronization of the pulses φH. The voltage level (Vout) corresponding to the charge accumulated in the FDA 112 is thus outputted from the buffer B to the CDS circuit 105.

It is understood from the above mentioned explanation that, during each of the red light emission period, the green light emission period, and the blue light emission period, the image formed on the CCD 111 is outputted as image information having n by n resolution from the CCD unit 110 to the CDS circuit 105.

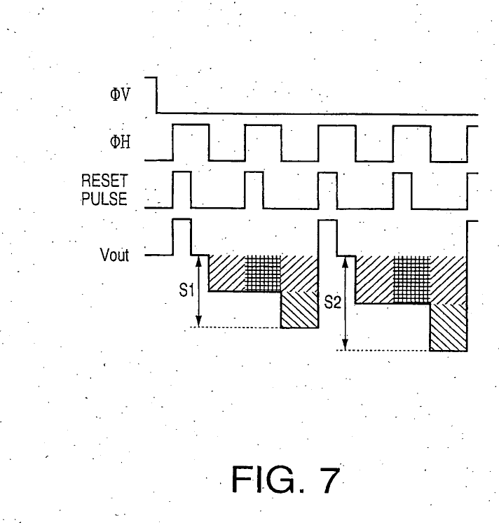

FIG. 6 is a timing chart showing the φV signal, the φH signal and the reset pulse during a period “d” (see FIG. 3) in the non-emission period following the UV light emission period. The driver circuit 104 switches the control of the CCD unit 110 in accordance with the select signal from the MPU 201. When the select signal is asserted, the control shown in FIGS. 6 and 7 is performed.

As shown in FIG. 6, a pair of pulses is periodically asserted as the φV signal. When the successive two pulses are inputted to the CCD unit 110, charge accumulated in each cell 111d is transferred vertically by two steps toward the horizontal transferring unit 111b. That is, when the successive two pulses φV are inputted to the CCD unit 110, charges accumulated in two cells 111d adjacently aligned in the vertical direction are added together in each cell 111e of the horizontal transferring unit 111b.

In the interval between two pairs of pulses φV, the φH signal and the reset pulse are asserted periodically.

FIG. 7 is a timing chart showing the φV signal, the φH signal and the reset pulse during a period “d′” in FIG. 6. As shown in FIG. 7, the φH signal is periodically asserted n times while the φV signal is low. The reset pulse rises in synchronization with rising of each odd-numbered pulse φH, and falls before the odd-numbered pulse φH falls.

The φH signal is asserted n times while the φV signal is low. Following the rising of each pulse φH, charge is transferred to the FDA 112, and the voltage level (S1,S2) corresponding to the charge accumulated in the FDA 112 is outputted from the FDA 112 until the next reset pulse is inputted to the FDA 112.

It is noted that the φH signal is asserted two times during a cycle of the reset pulse, charges of two cells 111e adjacently aligned in the horizontal transferring unit 111b are added together in the FDA 112. Thus, the voltage level (Vout) corresponding to the charge accumulated in the FDA 112 is outputted from the buffer B to the CDS circuit 105.

It is understood from the above mentioned explanation that, to the FDA 112, the sum of charges of four adjacent cells (2×2 matrix of cells) 111d is transferred. Accordingly, intensity of each pixel of the fluorescence image can be amplified fourfold in comparison with the normal image. The image formed on the CCD 111 is outputted as image information having n/2 by n/2 resolution from the CCD unit 110 to the CDS circuit 105.

As described above, according to the first embodiment of the invention, it is possible to amplify intensity of the image (i.e., the fluorescence image) captured by the emission of the UV light fourfold without using an expensive image intensifier.

Although in the above mentioned embodiment charges of 2×2 matrix of cells are added together to amplify the intensity of the image fourfold, the number of cells added together to amplify the fluorescence image can be changed.

For example, if a set of three pulses φV is outputted in place of the pair of pulses φV shown in FIG. 6, and if reset pulse is outputted each time the φH signal is asserted, the integration of 1×3 matrix of cells 111d can be attained. In this case, the intensity of the fluorescence image can be amplified threefold.

According to an aspect of the invention, the fluorescence image can be amplified at various kinds of scaling factors without using the expensive image intensifier.

Second Embodiment

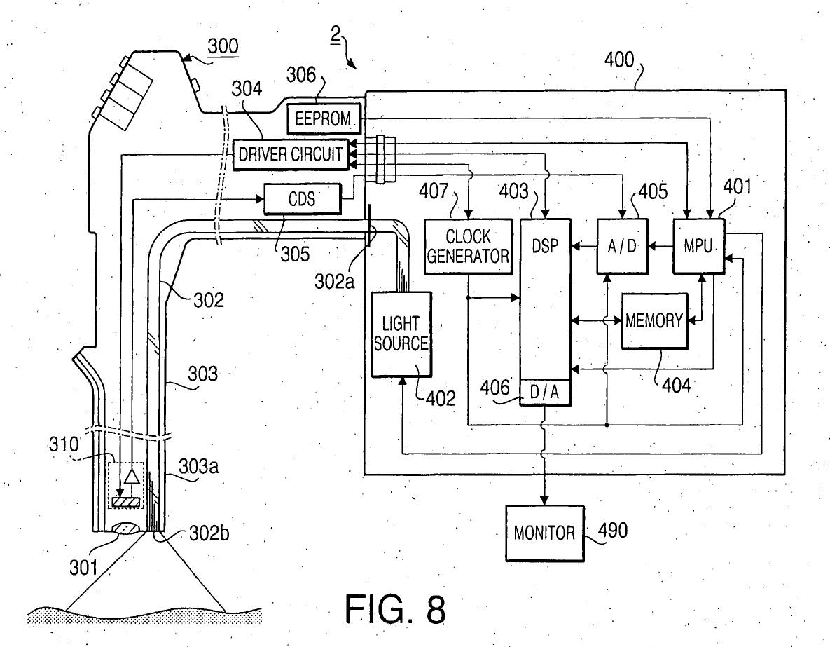

FIG. 8 shows a block diagram of an endoscope system 2 according to a second embodiment of the invention. As shown in FIG. 8, the general configuration of the endoscope system 2 is similar to that of the endoscope system 1 of the first embodiment. An endoscope (i.e., an electronic endoscope) 300 has a color CCD, and therefore emissions of three different kinds of light are not required to obtain color images in this embodiment.

As shown in FIG. 8, the endoscope system 2 includes the endoscope 300, an endoscope processor 400 and a monitor 490. The endoscope 300 includes an objective lens 301, a CCD unit 310 on which an image is formed by the objective lens 301, a fiber bundle (i.e., a light guide) 302, a driver circuit 304, a CDS (Correlated Double Sampling) circuit 305, and an EEPROM (electrically erasable programmable ROM) 306. The endoscope processor 400 includes a MPU (microprocessor unit) 401, a light source 402, a DSP (digital signal processor) 403, a memory 404, an A/D converter 405, a D/A converter 406 and a clock generator 407.

A proximal end portion 302a of the fiber bundle 302 is connected optically to the light source 402. A distal end 302b of the fiber bundle 302 is positioned at a distal end 303a of an insertion tube 303 of the endoscope 300. Light emitted from the light source 402 is transmitted through the fiber bundle 302 to illuminate the object from the distal end 302b of the fiber bundle 302. As described in detail later, the light source 402 is capable of emitting sequentially normal light and the excitation light to obtain color images and fluorescence images, respectively.

The objective lens 301 forms the image of the object which is illuminated by light from the light source 402 onto a light receiving surface of the CCD unit 310 driven by timing signals from the driver circuit 304.

The CCD unit 310 outputs an image signal in synchronization with the timing signals from the driver circuit 304. The image signal corresponding to one frame of image is outputted by the CCD unit during a time period including one emission period of light.

The CDS circuit 305 receives the image signal from the CCD unit 310 and processes the image signal to generate a CDS signal having an appropriate form for the A/D converter 405. The A/D converter 405 processes the CDS signal into digitized form. The digitized signal is transmitted from the A/D converter 405 to the DSP 403 in which image processing such as a γ-correction is performed on the digitized image signal to store the processed image signal as image data in the memory 404.

A frame of image data (captured by emission of normal light), and a frame of fluorescence image data (captured by emission of the excitation light) are stored in different areas of the memory 404.

The DSP 403 reads out the image data from the memory 404, and combines them to display the combined image. Then, the combined image data is processed by the D/A converter 406 having a video encoder to generate an analog video signal (e.g., an NTSC signal). Then, the video signal is transmitted to the monitor 490.

The above mentioned processes are performed under the control of the MPU 401 which executes various programs stored in, for example, a ROM embedded therein. That is, the MPU 401 controls the light source 402, the DSP 403, the memory 404, the A/D converter 405 and the D/A converter 406. For example, a sampling rate of the A/D converter 405 is determined by the MPU 401. The sampling rate determined by the MPU 401 is used by the A/D converter 405 to digitize the CDS signal at the sampling rate, and is also used by the DSP 403 to generate the image data. The MPU 401 uses an area in the memory 401 as a work memory.

The clock generator 407 generates a vertical synchronizing signal. The vertical synchronizing signal is sent to the driver circuit 304, the MPU 401, the DSP 403, and the A/D converter 405. The MPU 401 generates various control signals based on the vertical synchronizing signal to control the driver circuit 304, the light source 402, and the A/D converter 405.

Under the control of the MPU 401, the switching between the normal light emission and the excitation light emission, and an initiation of an image capturing operation of one frame are synchronized with the vertical synchronizing signal. The DSP 403 can determine which portion of the image signal sent from the endoscope 300 corresponds to one frame.

The driver circuit 304 provided in the endoscope 300 generates the timing signals based on the synchronizing signal from the clock generator 407 and sends the timing signals to the CCD unit 310 and the CDS circuit 305.

In the EEPROM 306 of the endoscope 300, information regarding the CCD unit 310 is stored. The information of the CCD unit 310 is read out by the MPU 401, and is used for the control of the DSP 403, the driver circuit 304, and the CDS circuit 305.

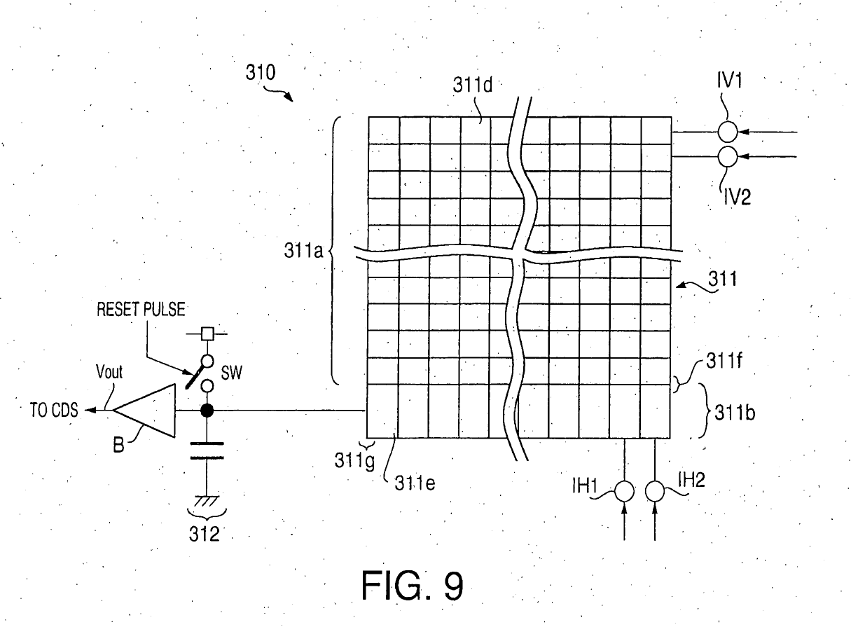

FIG. 9 shows a configuration of the CCD unit 310. The CCD unit 310 has the same architecture (i.e., a frame-transfer architecture) as the CCD unit 110 of the first embodiment shown in FIG. 2. Therefore, to some elements which are the same as those shown in FIG. 2, same numbers are assigned.

As shown in FIG. 9, the CCD unit 310 includes a color CCD (charge coupled device) 311, a Floating Diffusion Amplifier (i.e., a charge detection amplifier) 312, and buffer B. The CCD 311 includes a light receiving unit 311a on which the image is formed by the objective lens 301, a horizontal transferring CCD (HCCD) 311b, horizontal pulse input terminals IH1 and IH2, and vertical pulse input terminals IV1 and IV2.

The light receiving unit 311a has a plurality of cells 311d arranged in a form of M by N pixel matrix. That is, the light receiving unit 311a has M rows of cells 311d and N columns of sells 311d.

The HCCD 311b has N cells 311e arranged horizontally in a line and is adjacent to a side 311f of the light receiving unit 311a. The number of cells 311e in the HCCD 311b is equal to the number of cells 311d arranged horizontally along the side 311f.

The cells 311e in the HCCD 311b are respectively connected to the cells 311d arranged along the side 311f. One of the cells 311e located at one end (leftward end in FIG. 9) of the HCCD 311b is connected to the FDA 312.

The horizontal pulse input terminals IH1 and IH2, and the vertical pulse input terminals IV1 and IV2 are connected to the driver circuit 304, and receive the timing pulses from the driver circuit 304. Each of the vertical pulse input terminals IV1 and IV2 is connected to each cell 311d. Each of the horizontal pulse input terminals IH1 and IH2 is connected to each cell 311e in the HCCD 311b.

While the image is formed by the objective lens 301 on the cells 311d, charge is accumulated in each cell 311d. The amount of charge accumulated in each cell 311d depends on the accumulation period and a luminance of the optical image. Each time a pair of pulses are inputted to the vertical pulse input terminals IV1 and IV2, charge accumulated in each cell 311d is transferred vertically (i.e., downward in FIG. 9) to its neighboring cell toward the HCCD 311b. Also, charges accumulated in cells 311d arranged along the side 311f are respectively transferred to the cells 311e of the HCCD 311b.

In this embodiment, charge is transferred vertically based on the two-phase transferring technology. However, another technology, such as a single phase transferring technology, three-phase transferring technology, and four-phase transferring technology well known in the art, may be adopted to transfer charge vertically.

Each time pulses are inputted to the horizontal pulse input terminals IH1 and IH2, charge accumulated in each cell 311e is transferred horizontally (i.e., leftward in FIG. 9) to its neighboring cell. Also, charge accumulated in one of cells 311e located nearest to the FDA 312 is transferred to the FDA 312. In this embodiment, charge is transferred horizontally in HCCD 311b based on the two-phase transferring technology. However, another technology, such as a single phase transferring technology, three-phase transferring technology, and four-phase transferring technology well known in the art, may be adopted to transfer charge horizontally in the HCCD 311b.

The FDA 312 accumulates the charge transferred from the horizontal transferring CCD 311b until a next reset pulse is inputted thereto. The FDA 312 outputs a voltage level corresponding to the charge accumulated therein. The FDA 312 has the switch SW which is switched to ON or OFF by control of the reset pulse from the driver circuit 304.

When the reset pulse is inputted to the FDA 312, the charge accumulated therein is discharged, and thereafter next accumulation of charge starts. The voltage level generated by the FDA 312 is transmitted to the CDS circuit 305 through the buffer B. The buffer B may have the function of amplifying the voltage inputted therein. Thus, the image signal (i.e., Vout) generated by the CCD unit 310 is transmitted to the CDS circuit 305.

The CDS circuit 305 samples the image signal from the CCD unit 310 two times per one reset pulse (i.e., at a timing just after output of the reset pulse and at a timing just before output of the next reset pulse) to obtain a difference between amplitudes of the two values sampled between the reset pulse and the next reset pulse. The difference is outputted from the CDS circuit 305 to the A/D converter 405. According to the above mentioned operation of the CDS circuit 305, 1/f noise and reset pulse noise can be eliminated from the image signal outputted by the CDS circuit 305.

Although in this embodiment the CCD 311 has the frame-transfer architecture, a CCD having an interline architecture may be used in place of the CCD 311. FIG. 10 shows a CCD unit 1110 which adopts the interline architecture. The CCD unit 1110 includes a CCD 1111 having the interline architecture, a Floating Diffusion Amplifier (i.e., a charge detection amplifier) 1112, and a buffer B.

The CCD 1111 includes N columns of light receiving linens 1111a on which the image is formed by the objective lens 301, a horizontal transferring CCD (HCCD) 1111b, vertical transferring CCDs (VCCD) 1111h, horizontal pulse input terminals IH1 and IH2, and vertical pulse input terminals IV1 and IV2. As shown in FIG. 10, each light receiving line 1111a has M cells 1111d aligned vertically. That is, the CCD 1111 has the M by N matrix of cells 1111d for receiving light from the object.

Neighboring to each light receiving line 1111a, one vertical transferring CCD (VCCD) 1111h is located. The VCCD 1111h also includes M rows of cells 1111i aligned vertically. Cells 1111d in each light receiving line 1111a are respectively connected to the cells 1111i in the neighboring VCCD 1111h. Areas of the VCCDs 1111h are covered with optical shielding, and light from the object is not incident on the VCCDS 1111h. Therefore, only the cells 111d of light receiving lines 1111a receive light from the object.

The HCCD 1111b is located along one side 1111f of the CCD 1111. The HCCD 1111b has N cells 1111e which are aligned in a line horizontally and which respectively correspond to the VCCDs 1111h. One of the cells 1111e located at one end (leftward end) of the HCCD 1111b is connected to the FDA 1112.

As in the case of the CCD 311 having the frame-transfer architecture, the horizontal pulse input terminals IH1 and IH2, and the vertical pulse input terminals IV1 and IV2 are connected to the driver circuit 304, and receive the timing pulses from the driver circuit 304. Each of the vertical pulse input terminals IV1 and IV2 is connected to each cell 1111d. Each of the horizontal pulse input terminals IH1 and IH2 is connected to each cell 1111e of the HCCD 1111b.

While the image is formed by the objective lens 301 on the cells 1111d, charge is accumulated in each cell 1111d. The amount of charge accumulated in each cell 1111d depends on the accumulation period and a luminance of the optical image. Each time pulses are inputted to the vertical pulse input terminals IV1 and IV2, charge transferred from each cell 1111d to each cell 1111i is transferred vertically (i.e., downward in FIG. 10) to its neighboring cell toward the HCCD 1111b. Also, charges accumulated in cells 1111i arranged along the side 111f are respectively transferred to the cells 1111e of the HCCD 1111b.

In this example shown in FIG. 10, charge is transferred vertically based on the two-phase transferring technology. However, another technology, such as a single phase transferring technology, three-phase transferring technology, and four-phase transferring technology well known in the art, may be adopted to transfer charge vertically.

Each time a pair of pulses are inputted to the horizontal pulse input terminals IH1 and IH2, charge accumulated in each cell 1111e is transferred horizontally (i.e., leftward in FIG. 10) to its neighboring cell. Also, charge accumulated in one of cells 1111e located nearest to the FDA 1112 is transferred to the FDA 1112. In this example, charge is transferred horizontally based on the two-phase transferring technology. However, another technology, such as a single phase transferring technology, three-phase transferring technology, and four-phase transferring technology well known in the art, may be adopted to transfer charge horizontally.

As described above, the CCD unit 1110 can be controlled in the same way as that of the CCD unit 310. Functions of the FDA 1112 and the buffer B are the same as those shown in FIG. 9, detailed explanation thereof will not be repeated.

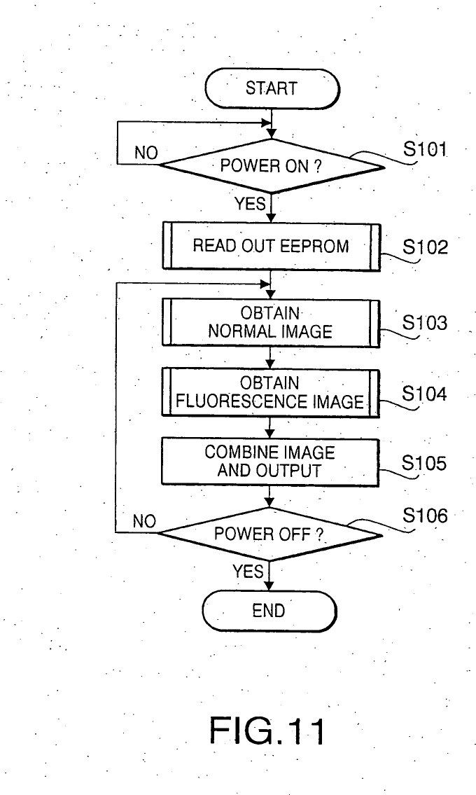

Hereafter, an image processing performed under control of the MPU 401 provided in the endoscope processor 400 will be described. As described below, images for normal observation (i.e., color images) and images for fluorescent observation (i.e., fluorescence images) are obtained in the image processing; FIG. 11 is a flowchart (a main flow) showing the image processing. A program of the image processing is stored in the embedded ROM and is read out by the MPU 401.

Firstly, the MPU 401 determines whether power of the endoscope 300 is ON or OFF (step S101). When the power of the endoscope 300 is OFF (S101:NO), the MPU 401 repeats the step S101. When the power of the endoscope 300 is ON (S101:YES), control proceeds to step S102.

In step S102, the MPU 401 reads out information from the EEPROM 306 and assigns various parameters to variables. Next, in step S103, the image for the normal observation is obtained. Next, in step S104, the image for the fluorescent observation is obtained.

In step S105, the image for normal observation obtained in step S103 and the image for the fluorescent observation obtained in step S104 are combined. Then, the combined image is converted to the video signal and is outputted to the monitor 490. Next, in step S106, the MPU 401 determines whether the power of the endoscope 300 is OFF or ON.

When the endoscope 300 is ON (S106:NO), control returns to step S103 to obtain a next frame of the image. When the endoscope 300 is OFF (S106:YES), the image processing terminates. According to the image processing shown FIG. 11, both of the image for the normal observation and the image for the fluorescent observation can be obtained and are displayed on the monitor 490.

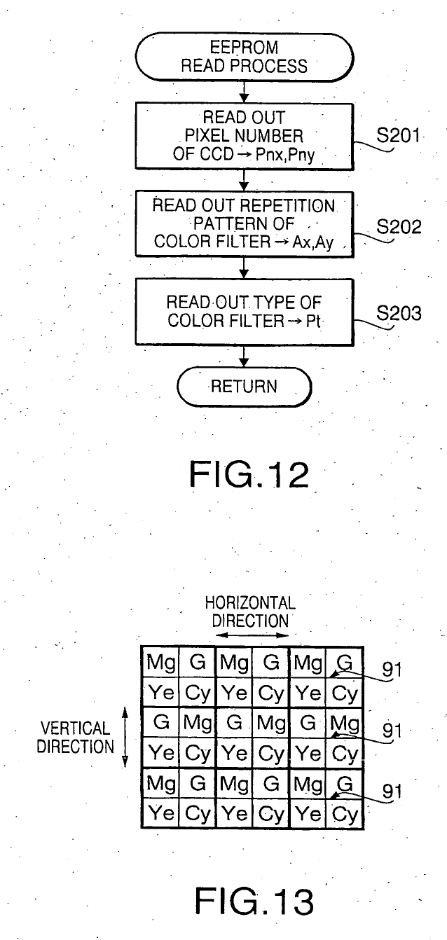

FIG. 12 is a flowchart showing an EEPROM read process performed in step S102 of FIG. 11.

Firstly, in step S201, the MPU 401 obtains the number of cells 311d provided in the light receiving unit 311a. More specifically, the MPU 401 obtains the number of cells 311d in one row (i.e., the cells 311d aligned in the horizontal direction) and the number of cells 311d in one column (i.e., the cells 311d aligned in the vertical direction) from the EEPROM 306. The MPU 401 assigns the number of cells 311d in one row to a variable Pnx, and assigns the number of cells 311d in one column to a variable Pny.

As described above, in this embodiment, the cells 311d aligned in M by N matrix. The number N is assigned to the Pnx and the number M is assigned to the Pny.

On cells 311d of the color CCD 311, a color filter is formed. FIG. 13 shows an example of color filter array patterns. The color filter shown in FIG. 13 has repetitions of a color group 91 which has a green color element G and complementary color elements of magenta Mg, yellow Ye and cyan Cy. When the image for the normal observation is obtained, one pixel of the image is formed by four cells 311d corresponding to four filter elements of Mg, Ye, Cy and G in one group 91.

In step S202, information concerning a repetition pattern of the filter elements of the color filter is read out from the EEPROM 306. More specifically, the information concerning the repletion pattern includes the number of filter elements arranged in the horizontal direction in one group 91, and the number of filter elements arranged in the vertical direction in one group 91.

In the case of the color filter shown in FIG. 13, the number of filter elements arranged in the horizontal direction in one group 91 is 2, and the number of filter elements arranged in the vertical direction in one group 91 is 2. These obtained numbers (2, 2) are stored in variables (AX, Ay), respectively.

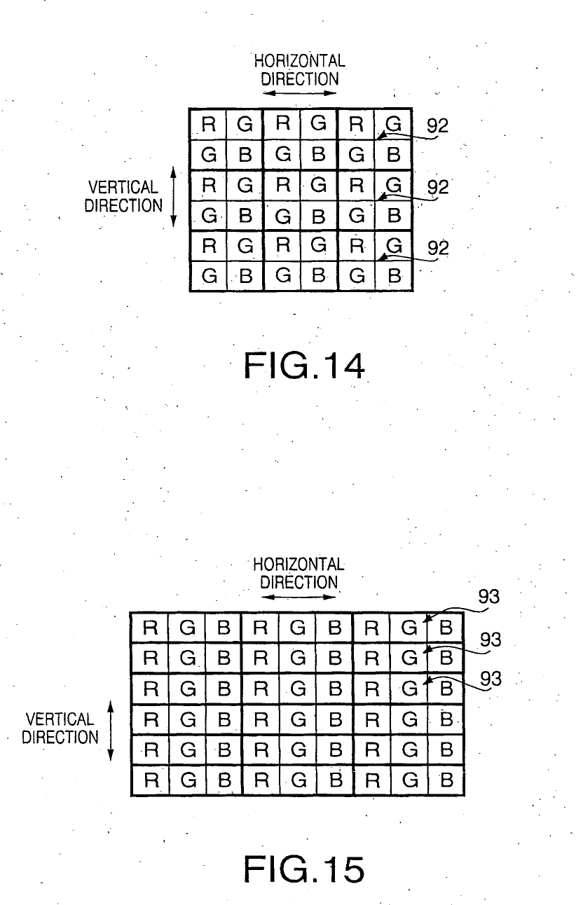

FIG. 14 shows another example of the color filter array pattern. The color filter shown in FIG. 14 has repetitions of a color group 92 which has two green color elements G, a red color element R and a blue color element B. When the image for the normal observation is obtained, one pixel of the image is formed by four cells 311d corresponding to four filter elements of R, G, G and B in one group 92. In the case of the color filter shown in FIG. 14, the number of filter elements arranged in the horizontal direction in one group 92 is 2, and the number of filter elements arranged in the vertical direction in one group 92 is 2. Therefore, these obtained numbers (2, 2) are assigned to variables (AX, Ay), respectively.

FIG. 15 shows another example of the color filter array pattern. The color filter shown in FIG. 15 has repetitions of a color group 93 which has a red color elements R, a green color element G and a blue color element B. The color filter array pattern shown in FIG. 15 is frequently called a RGB stripe array.

When the image for the normal observation is obtained, one pixel of the image is formed by three cells 311d corresponding to three filter elements of R, G and B in one group 93. In the case of the color filter shown in FIG. 15, the number of filter elements arranged in the horizontal direction in one group 93 is 3, and the number of filter elements arranged in the vertical direction in one group 93 is 1. Therefore, these obtained numbers (3, 1) are assigned to variables (AX, Ay), respectively.

After the information concerning the repetition pattern of the filter elements is obtained in step S202, control proceeds to step S203. In step S203, the MPU 401 obtains an ID number indicating the type of the color filter array pattern of the color filter in the color CCD 310 from the EEPROM 306.

For example, the ID number of the type of the color filter array pattern is zero when the color filter shown in FIG. 13 is used, the ID number of the type of the color filter array pattern is 1 when the color filter shown in FIG. 14 is used, and the ID number of the type of the color filter array pattern is 2 when the color filter shown in FIG. 15 is used. The ID number of the type of the color filter array pattern is stored in a variable Pt.

After step S203 is processed, control returns to step 103 of the main flow (FIG. 11).

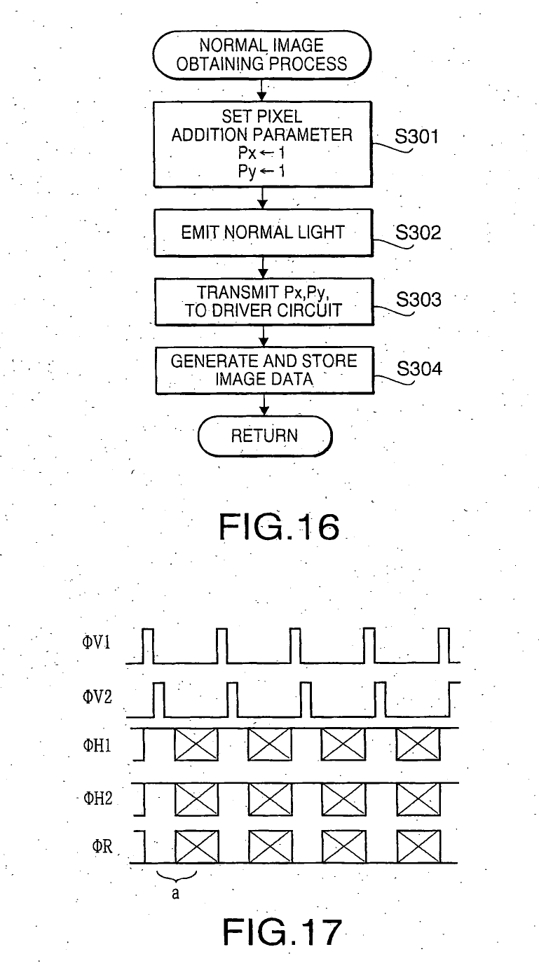

FIG. 16 is a flowchart showing a normal image obtaining process performed in step S103 of the main flow (FIG. 11). Firstly, in step S301, pixel addition parameters are assigned to Px and Py (i.e., 1 is assigned to each of the variables Px and Py). Next, in step S302, normal light (i.e., white light) is emitted by the light source 402.

In step S303, the values of Px and Py are sent to the driver circuit 304. The driver circuit 304 uses the Px and Py to generate timing signals to be inputted to the CCD unit 310 and the CDS circuit 305. By performing the step S303, one frame of the image signal is outputted by the CDS circuit 305 to the A/D converter 405.

In step S304, the MPU 401 controls the A/D converter 405 and the DSP 403 to convert the image signal to image data. The image data is then stored in the memory 404. Various parameters obtained in step S102 in the main flow are used in the conversion step S304. Hereafter, the digitized image signal corresponding to one frame is represented by pixel data P(x,y) where 0≦x<Pnx, 0≦y<Pny.

The DSP 403 converts the pixel data P(x,y) to red, green and blue component image data of R(x,y), G(x y) and B(x,y). More specifically, R(x,y) represents a red component intensity value of a pixel having coordinates (x,y). G(x,y) represents a green component intensity value of a pixel having coordinates (x,y). Further, B(x,y) represents a blue component intensity value of a pixel having coordinates (x,y).

When the color filter shown in FIG. 13 is used (i.e., if Pt=0), the DSP 403 determines R(x,y), G(x,y) and B(x,y) according to the following equation (1):

$$( \begin{matrix}R ( x , y ) \\ G ( x , y ) \\ B ( x , y )\end{matrix} ) = ( \begin{matrix}Kr1 & Kr2 & Kr3 & Kr4 \\ Kg1 & Kg2 & Kg3 & Kg4 \\ Kb1 & Kb2 & Kb3 & Kb4\end{matrix} ) * ( \begin{matrix}P ( x , y ) \\ P ( x + 1 , y ) \\ P ( x , y + 1 ) \\ P ( x + 1 , y + 1 )\end{matrix} ) \qquad ( 1 )$$

where 0≦x<(Pnx−1), 0≦y<(Pny−1).

In the equation (1), matrix factors Kr1, Kr2, Kr3, Kr4, Kg1, Kg2, Kg3, Kg4, Kb1, Kb2, Kb3, Kb4 are adjusted in accordance with luminosity characteristics. Also, the matrix factors change depending on the type of the color filter. That is, the matrix factors change depending on the types of the filter elements on the cells corresponding to P(x,y), P(x+1,y), P(x,y+1) and P(x+1, y+1).

The digital image data of one pixel is thus obtained from charges accumulated in four cells 311d according to the equation (1).

When the color filter shown in FIG. 14 is used (i.e., if Pt=1), the DSP 403 determines R(x,y), G(x,y) and B(x,y) according to the following equations (2), given that 0≦x<(Pnx−1), 0≦y<(Pny−1). In the following equations, the green filter element (G) adjoining to the red filter element (R) in the horizontal direction is defined as a Gr filter element, and green filter element (G) adjoining to the blue filter element (B) in the horizontal direction is defined as a Gb filter element.

If the red filter element (R) is located on the cell corresponding to the P(x,y), R(x,y), G(x,y) and B(x,y) are determined as follows.

R(x,y)=P(x,y)

G(x,y)=[P(x,y−1)+P(x−1,y)+P(x+1,y)+P(x,y+1)]/4

B(x,y)=[P(x−1,y−1)+P(x+1,y−1)+P(x−1,y+1)+P(x+1,y+1)]/4

If the Gr filter element is located on the cell corresponding to the P(x,y), R(x,y), G(x,y) and B(x,y) are determined as follows.

R(x,y)=[P(x−1,y)+P(x+1,y)]/2

G(x,y)=P(x,y)

B(x,y)=[P(x,y−1)+P(x,y+1)]/2

If the Gb filter element is located on the cell corresponding to the P(x,y), R(x,y), G(x,y) and B(x,y) are determined as follows.

R(x,y)=[P(x,y−1)+P(x,y+1)]/2

G(x,y)=P(x,y)

B(x,y)=[P(x−1,y)+P(x+1,y)]/2

If the blue filter element (B) is located on the cell corresponding to the P(x,y), R(x,y), G(x,y) and B(x,y) are determined as follows.

R(x,y)=[P(x−1,y−1)+P(x+1,y−1)+P(x−1,y+1)+P(x+1,y+1)]/4

G(x,y)=[P(x,y−1)+P(x−1,y)+P(x+1,y)+P(x,y+1)]/4

B(x,y)=P(x,y) (2) (2)

The digital image data of one pixel when the color filter shown in FIG. 14 is used is thus obtained from charges accumulated in four cells 311d according to the above equations.

When the color filter (RGB stripe array) shown in FIG. 15 is used (i.e., if Pt=2), the DSP 403 determines R(x,y), G(x,y) and B(x,y) according to the following equations (3), given that 0≦x<(Pnx−1), 0≦y<(Pny−1).

If the red filter element (R) is located on the cell corresponding to the P(x,y), R(x,y), G(x,y) and B(x,y) are determined as follows.

R(x,y)=P(x,y)

G(x,y)=P(x+1,y)

B(x,y)=P(x−1,y)

If the green filter element (G) is located on the cell corresponding to the P(x,y), R(x,y), G(x,y) and B(x,y) are determined as follows.

R(x,y)=P(x−1,y)

G(x,y)=P(x,y)

B(x,y)=P(x+1,y)

If the blue filter element (B) is located on the cell corresponding to the P(x,y), R(x,y), G(x,y) and B(x,y) are determined as follows.

R(x,y)=P(x+1,y)

G(x,y)=P(x−1,y)

B(x,y)=P(x,y) (3)

The digital image data of one pixel when the color filter shown in FIG. 15 is used is thus obtained from charges accumulated in three cells 311d according to the above equations.

After the above mentioned calculation is performed in step S304 of FIG. 16, the normal image obtaining process terminates, and control returns to step S104 of the main flow (FIG. 11).

Next, operation of the driver circuit 304, the CDS 305 and CCD unit 310 when the MPU 401 sends the Px and Py to the driver circuit 304 in step S303 of FIG. 16 will be explained. FIG. 17 is a timing chart showing the timing signals outputted by the driver circuit 304 when the driver circuit 304 receives the Px and Py from the MPU 401. That is, the timing chart shows operation when the normal image is obtained.

As shown in FIG. 17, the driver circuit 304 outputs a φV1 signal, a φV2 signal, a φH1 signal, a φH2 signal and a φR signal (i.e., the reset pulse). The φV1 signal and the φV2 signal are respectively inputted to the vertical pulse input terminals IV1 and IV2 of the CCD unit 310. The φH1 signal and the φH2 signal are respectively inputted to the horizontal pulse input terminals IH1 and IH2. The φR signal is inputted to the switch SW of the FDA 312.

As shown in FIG. 17, when a pair of successive one φV1 pulse and one φV2 pulse is inputted to the input terminals IV1 and IV2, charge accumulated in each cell 311d is transferred vertically (i.e., downward in FIG. 9) to its neighboring cell 311d. Then, by respectively inputting the φH1 pulses and φH2 pulses to the input terminals IH1 and IH2, charge accumulated in each cell 311e in the HCCD 311b is outputted from the HCCD 311b to the FDA 312. Further, by inputting the φR pulses to the CCD unit 310, the image signal is sent from the CCD unit 310 to the CDS circuit 305.

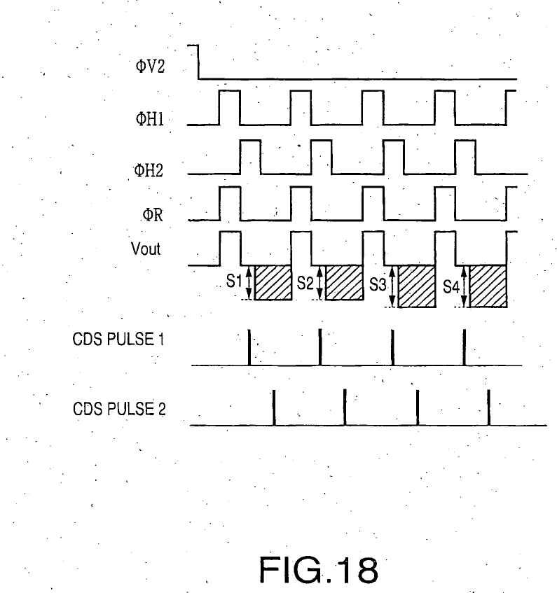

FIG. 18 is a timing chart showing the timing within a timing period “a” of FIG. 17 in detail. As shown in FIG. 18, the φR pulses are inputted to the CCD unit 310 in synchronization with the φH1 pulses. Also, in FIG. 18, CDS pulses 1 and 2 which are sent from the driver circuit 304 to the CDS circuit 305 are indicated.

The CDS circuit 305 samples the image signal from the CCD unit 310 when the CDS pulse 1 is inputted thereto and when the CDS pulse 2 is inputted thereto. Then the CDS circuit 305 obtains a difference between amplitude of the image signal when the CDS pulse 1 is inputted and amplitude of the image signal when the CDS pulse 2 is inputted.

More specifically, the CDS pulse 1 is asserted just after the falling edge of the φR pulse, and the CDS pulse 2 is asserted just before the rising edge of the next φR pulse. Synchronizing with the falling edge of the φH2 pulse, charge accumulated in each cell 311e of the HCCD 311b is transferred to its neighboring cell. The FDA 312 accumulates charge from the cell 311e of the HCCD 311b. The FDA 312 outputs the voltage level corresponding to the charge accumulated therein. Consequently, a signal Vout (i.e., the image signal) corresponding to the charge accumulated in the FDA 312 is outputted from the buffer B to the CDS circuit 305.

According to the above mentioned operation of the CCD unit 310 and the CDS circuit 305, the difference between amplitude of the image signal sampled when the CDS pulse 1 is asserted and amplitude of the image signal when the CDS pulse 2 is asserted appropriately indicates the voltage level corresponding to charge accumulated in the FDA 312.

That is, the CDS circuit 305 outputs voltage levels respectively corresponding to voltage levels of S12, S2, S3 and S4 shown in FIG. 18. As shown in FIG. 18, one φR pulse is asserted per one φH1 pulse. Therefore, each of voltage levels S1, S2, S3 and S4 corresponds to charge accumulated in one cell 311e.

As describe above, Charge accumulated in one cell 311d is transferred to each cell 311e of the HCCD 311b when the pair of signals φV1 and φV2 is asserted. Therefore, each of the voltage levels of S1, S2, S3 and S4 shown in FIG. 18 corresponds to one cell 311d of the light receiving unit 311a.

Consequently, the CDS circuit 305 sequentially outputs voltage levels respectively corresponding to charges in the cells 311d.

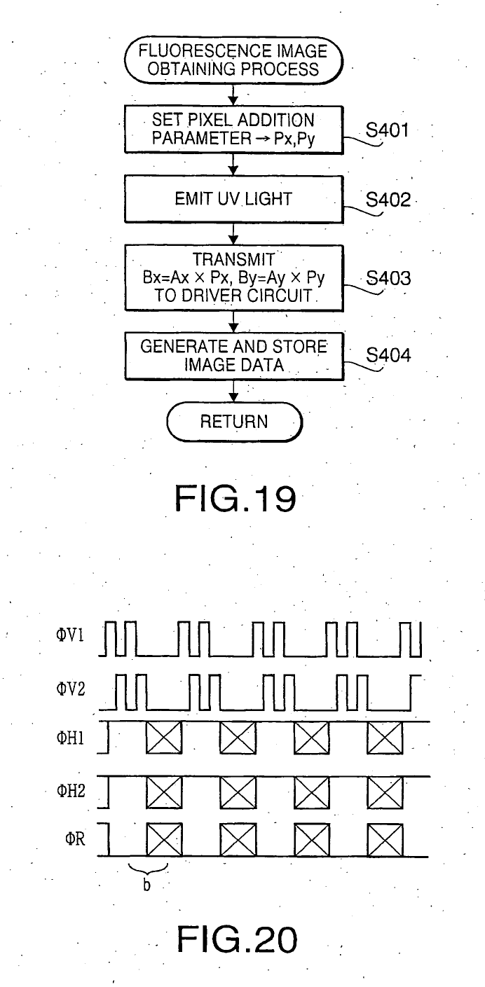

FIG. 19 is a flowchart showing in detail a process of obtaining the image for the fluorescence observation (hereafter, referred to as a fluorescence image obtaining process) performed in step S104 of FIG. 11.

Firstly, in step S401, pixel addition parameters are assigned to Px and Py. The pixel addition parameters represent the number of pixels of the normal image to be added together for generating one pixel of the fluorescence image.

For example, if Px=2 and Py=3, intensities of two cell groups (e.g., two color groups 91 in FIG. 13) aligned in the horizontal direction and intensities of three cell groups aligned in the vertical direction are added together, and the sum is used as an intensity of one pixel of the fluorescence image. That is, 2 by 3 pixel groups are used to generate one pixel of the fluorescence image. The pixel addition parameters may be set by a user to the endoscope processor 400 for example by user operation.

As described above, one color group (e.g., the color group 91 in FIG. 13) of the color filter has different types of color filter elements. It is noted that the different types of color filter elements have different transmissivity. Therefore, in order to match a gray scale of the fluorescence image to a real image of the object, the number of cells 311d used for generation of one pixel of the fluorescence image has to be integral multiples of the number of cells 311d included in one color group.

That is, the number of cells 311d in the horizontal direction used to generate one pixel of the fluorescence image has to be integral multiples of the number of cells 311d in the horizontal direction used to generate one pixel of the normal image. Also, the number of cells 311d in the vertical direction used to generate one pixel of the fluorescence image has to be integral multiples of the number of cells 311d in the vertical direction used to generate one pixel of the normal image.

In step S402, the MPU 401 controls the light source 402 to emit UV light (i.e., the excitation light). Next, in step S403, the product of Ax and Px is assigned to a variable Bx, and the product of Ay and Py is assigned to a variable By. Then, the MPU 401 sends the variables Bx and By to the driver circuit 304. The driver circuit 304 generates the timing signals based on the variables Bx and By. By performing the step S403, a frame of the image signal is outputted by the CDS circuit 305.

Next, in step S404, the MPU 401 controls the A/D converter 405 and the DSP 403 to convert the image signal from the CDS circuit 305 to image data. The image data is then stored in the memory 404. The various parameters obtained by the MPU 401 in step S102 of the main flow are used in the conversion process in step S404.

Hereafter, a frame of the digitized signal generated by the A/D converter 405 is represented by P′(x,y) having the pixel number of Pnx/Bx×Pny/By (where 0≦x<Pnx/Bx, 0≦y<Pny/By). The DSP 403 converts the P′(x,y) to image data F(x,y). The F(x,y) represents an intensity value of one pixel data of the fluorescence image having the horizontal coordinate x and the vertical coordinate y.

The DSP 403 determines the F(x,y) according to the following equations.

0≦x<Pnx/Bx−1

0≦y<Pny/By−1

F(x,y)=P′(x,y)

After the image data is obtained in step S404, the fluorescence image obtaining process terminates. Then, control returns to step S105 of the main flow.

The operation of the driver circuit 304, the CCD unit 310 and the CDS circuit 305 when the Bx and By are transmitted from the MPU 401 to the driver circuit 304 will be described below. FIG. 20 is a timing chart showing the timing signals outputted by the driver circuit 304 when the Bx and By are transmitted from the MPU 401 to the driver circuit 305.

As show in FIG. 20, the driver circuit 304 outputs successive “By φV1 pulses (i.e., 2 pulses in the example shown in FIG. 13) and successive “By” φV2 pulses while the φH1 and φH2 are high in order to transfer charge accumulated in each cell 311d vertically (i.e., downward in FIG. 10) by “By” steps (i.e., 2 steps in the example shown in FIG. 13) toward the HCCD 311b. After the two successive φV1 pulses and the two successive φV2 pulses are asserted, φH1 pulses and φH2 pulses are asserted successively to transfer charges accumulated in cells 311e to the FDA 312, and φR pulses are also asserted to transmit the image signal from the CCD unit 310 to the CDS circuit 305.

By inputting the φV1 pulses and φV2 pulses as described above, charges of “By” cells 311d (i.e., two cells 311d in the example shown in FIG. 13) aligned in the vertical direction are added together in each cell 311e of the HCCD 311b.

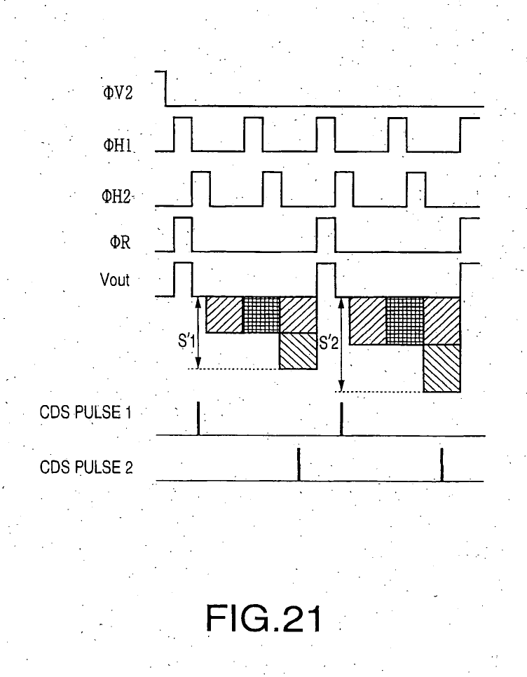

FIG. 21 is a timing chart showing in detail the timing of the pulse φH1, pulse φH2, pulse φR, Vout (i.e., the image signal), the CDS pulse 1 and the CDS pulse 2 during a time period “b” indicated in FIG. 20. As shown in FIG. 21, the pulse φR is asserted one time while pulses φH1 are asserted Bx times (i.e., two times in an example of FIG. 21). Each pulse φR is asserted in synchronization with its corresponding pulse φH1.

When the φH2 pulse falls, charge accumulated in each cell 311e of the HCCD 311b is transferred leftward to its neighboring cell 311e. The φH2 pulse is asserted Bx times (i.e., two times in the example shown in FIG. 14) during an interval between two successive φR pulses. The FDA 312 starts to accumulate charge from the HCCD 311b after the φR pulse falls. Therefore, in the interval between two pulses φR, charges of Bx cells 311e (i.e., charges of two cells 311e in the example shown in FIG. 21) are added together into the FDA 312, and are converted to a voltage level by the FDA 312.

The CDS pulse 1 is asserted just after the falling edge of the φR pulse, while the CDS pulse 2 is asserted just before the rising edge of the next φR pulse. Consequently, the CDS circuit 305 outputs voltage levels respectively corresponding to amplitudes S′1 and S′2 indicated in FIG. 21.

It is understood from the above explanation that each of the amplitude S′1 and amplitude S′2 corresponds to charges accumulated in successive Bx cells 311e in the HCCD 311b. In addition, as described above, charges of By (i.e., two cells) 311d aligned in the vertical direction are added together in each cell 311e of the HCCD 311b. Therefore, each of the amplitude S′1 and amplitude S′2 corresponds to the sum of charges accumulated in cells 311d arranged in By×Bx matrix.

As described above, Bx is the product of Ax and Px, and By is the product of Ay and Py. Therefore, by inputting the above mentioned timing pulses to the CCD unit 310 and the CDS circuit 305, the CDS circuit 305 outputs sequentially voltages levels, each of which corresponds to integral multiple of one pixel of the normal image.

According to the embodiment of the invention, the image formed on the light receiving unit 311a can be amplified Bx×By times.

Third Embodiment

Hereafter, a third embodiment of the invention will be described. An endoscope system according to the third embodiment has the same hardware configuration as the second embodiment shown in FIG. 8.

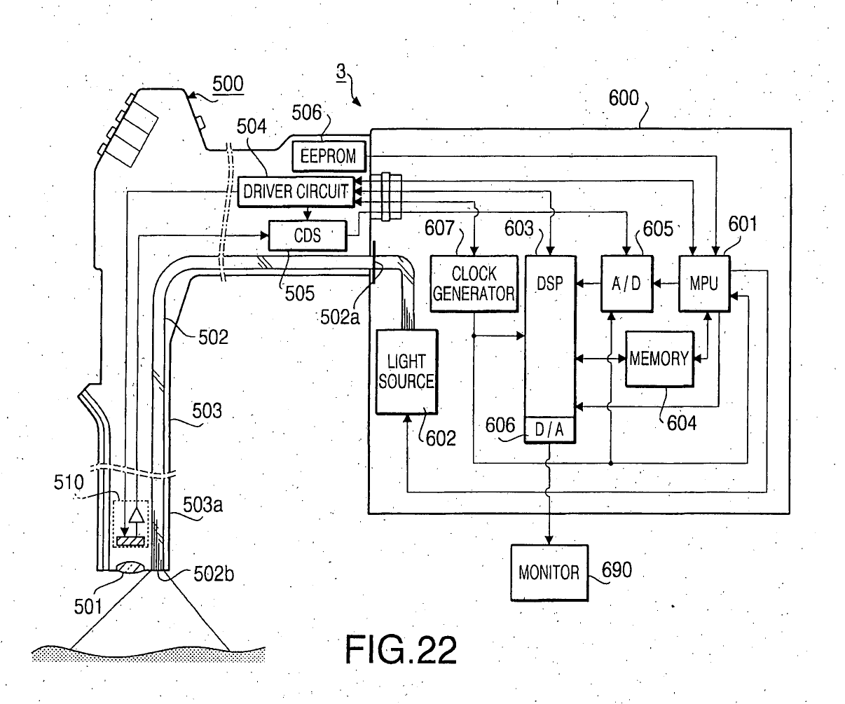

FIG. 22 shows a block diagram of the endoscope system 3 according to the third embodiment of the invention. The endoscope system includes an endoscope (i.e., an electronic endoscope) 500, an endoscope processor 600 and a monitor 690.

The endoscope 500 includes an objective lens 501, a CCD unit 510 on which an image is formed by the objective lens 501, a fiber bundle (i.e., a light guide) 502, a driver circuit 504, a CDS (Correlated Double Sampling) circuit 505, and an EEPROM (electrically erasable programmable ROM) 506. The endoscope processor 600 includes a MPU (microprocessor unit) 601, a light source 602, a DSP (digital signal processor) 603, a memory 604, an A/D converter 605, a D/A converter 606 and a clock generator 607.

A proximal end portion 502a of the fiber bundle 502 is connected optically to the light source 602. A distal end 502b of the fiber bundle 502 is positioned at a distal end 503a of an insertion tube 503 of the endoscope 500. Light emitted from the light source 602 is transmitted through the fiber bundle 502 to illuminate the object from the distal end 502b of the fiber bundle 502. As described in detail later, the light source 602 is capable of emitting sequentially normal light and the excitation light to obtain color images and fluorescence images, respectively.

The objective lens 501 forms the image of the object which is illuminated by light from the light source 602 onto a light receiving surface of the CCD unit 510 driven by timing signals from the driver circuit 504.

The CCD unit 510 outputs an image signal in synchronization with the timing signals from the driver circuit 504. The image signal corresponding to one frame of image is outputted by the CCD circuit during a time period including one emission period of light.

The CDS circuit 505 receives the image signal from the CCD unit 510 and processes the image signal to generate a CDS signal having an appropriate form for the A/D converter 605. The A/D converter 605 processes the CDS signal into digitized form. The digitized signal is transmitted from the A/D converter 605 to the DSP 603 in which image processing such as a γ-correction is performed on the digitized image signal to store the processed image signal as image data in the memory 604.

A frame of image data (captured by emission of normal light (i.e., white light)), and a frame of fluorescence image data (captured by emission of the excitation light) are stored in different areas of the memory 604.

The DSP 603 reads out the image data from the memory 604, and combines them to display the combined image. Then, the combined image data is processed by the D/A converter 606 having a video encoder to generate an analog video signal (e.g., an NTSC signal). Then, the video signal is transmitted to the monitor 690.

The above mentioned processes are performed under the control of the MPU 601 which executes various programs stored in, for example, a ROM embedded therein. That is, the MPU 601 controls the light source 602, the DSP 603, the memory 604, the A/D converter 605 and the D/A converter 606. For example, a sampling rate of the A/D converter 605 is determined by the MPU 601. The sampling rate determined by the MPU 601 is used by the A/D converter 605 to digitize the CDS signal at the sampling rate, and is also used by the DSP 603 to generate the image data. The MPU 601 uses an area in the memory 401 as a work memory.

The clock generator 607 generates a vertical synchronizing signal. The vertical synchronizing signal is sent to the driver circuit 504, the MPU 601, the DSP 603, and the A/D converter 605. The MPU 601 generates various control signals based on the vertical synchronizing signal to control the driver circuit 504, the light source 602, and the A/D converter 605.

Under the control of the MPU 601, the switching between the normal light emission and the excitation light emission, and an initiation of an image capturing operation of one frame are synchronized with the vertical synchronizing signal. The DSP 603 can determine which portion of the image signal sent from the endoscope 500 corresponds to one frame.

The driver circuit 504 provided in the endoscope 500 generates the timing signals based on the synchronizing signal from the clock generator 607 and sends the timing signals to the CCD unit 510 and the CDS circuit 505.

In the EEPROM 506 of the endoscope 500, information regarding the CCD unit 510 is stored. The information of the CCD unit 510 is read out by the MPU 601, and is used for the control of the DSP 603, the driver circuit 604, and the CDS circuit 505.

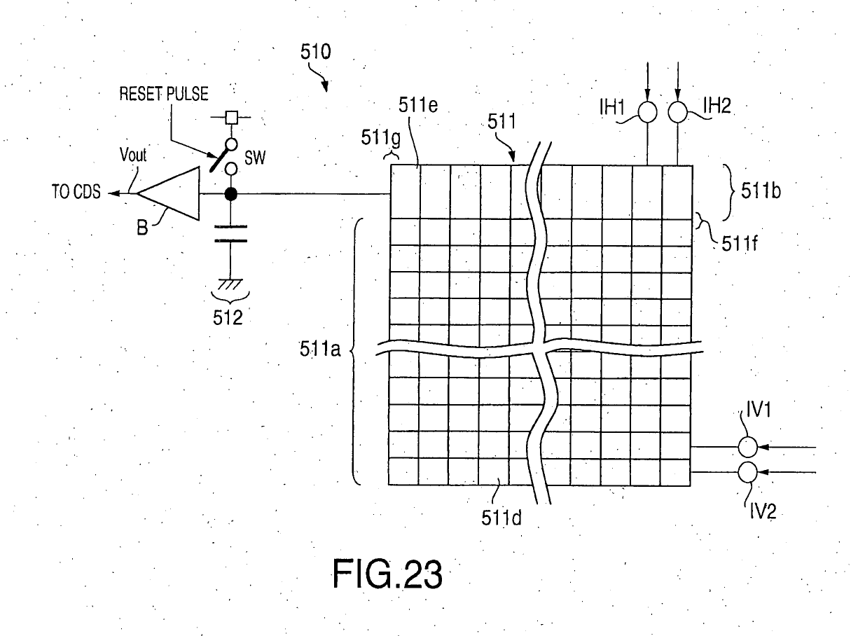

FIG. 23 shows a configuration of the CCD unit 510. The CCD unit 510 has the same architecture (i.e., the frame-transfer architecture) as the CCD unit 310 of the second embodiment shown in FIG. 9. Therefore, to some elements which are the same as those shown in FIG. 9, same numbers are assigned.

As shown in FIG. 23, the CCD unit 510 includes a color CCD (charge coupled device) 511, a Floating Diffusion Amplifier (i.e., a charge detection amplifier) 512, and buffer B. The CCD 511 includes a light receiving unit 511a on which the image is formed by the objective lens 501, a horizontal transferring CCD (HCCD) 511b, horizontal pulse input terminals IH1 and IH2, and vertical pulse input terminals IV1 and IV2.

The light receiving unit 511a has a plurality of cells 511d arranged in a form of M by N pixel matrix. That is, the light receiving unit 511a has M rows of cells 511d and N columns of sells 511d.

The HCCD 311b has N cells 311e arranged horizontally in a line and is adjacent to a side 511f of the light receiving unit 511a. The number of cells 511e in the HCCD 511b is equal to the number of cells 311d arranged horizontally along the side 511f.

The cells 511e in the HCCD 511b are respectively connected to the cells 511d arranged along the side 511f. One of the cells 511e located at one end (leftward end in FIG. 23) of the HCCD 311b is connected to the FDA 512.

The horizontal pulse input terminals IH1 and IH2, and the vertical pulse input terminals IV1 and IV2 are connected to the driver circuit 504, and receive the timing pulses from the driver circuit 504. Each of the vertical pulse input terminals IV1 and IV2 is connected to each cell 511d. Each of the horizontal pulse input terminals IH1 and IH2 is connected to each cell 511e in the HCCD 511b.

While the image is formed by the objective lens 501 on the cells 511d, charge is accumulated in each cell 511d. The amount of charge accumulated in each cell 511d depends on the accumulation period and a luminance of the optical image. Each time a pair of pulses are inputted to the vertical pulse input terminals IV1 and IV2, charge accumulated in each cell 511d is transferred vertically into its neighboring cell toward the HCCD 511b. Also, charges accumulated in cells 511d arranged along the side 511f are respectively transferred to the cells 511e of the HCCD 511b.

In this embodiment, charge is transferred vertically based on the two-phase transferring technology. However, another technology, such as a single phase transferring technology, three-phase transferring technology, and four-phase transferring technology well known in the art, may be adopted to transfer charge vertically.

Each time a pair of pulses are inputted to the horizontal pulse input terminals IH1 and IH2, charge accumulated in each cell 511e is transferred horizontally (i.e., leftward in FIG. 23) to its neighboring cell. Also, charge accumulated in one of cells 511e located nearest to the FDA 512 is transferred to the FDA 512. In this embodiment, charge is transferred horizontally in the HCCD 511b based on the two-phase transferring technology. However, another technology, such as a single phase transferring technology, three-phase transferring technology, and four-phase transferring technology well known in the art, may be adopted to transfer charge horizontally in the HCCD 511b.

The FDA 512 accumulates the charge transferred from the HCCD 511b until a next reset pulse is inputted thereto. The FDA 512 outputs a voltage level corresponding to the charge accumulated therein. The FDA 512 has the switch SW which is switched to ON or OFF by control of the reset pulse from the driver circuit 504.

When the reset pulse is inputted to the FDA 512, the charge accumulated therein is discharged, and thereafter next accumulation of charge starts. The voltage level generated by the FDA 512 is transmitted to the CDS circuit 505 through the buffer B. The buffer B may have the function of amplifying the voltage inputted therein. Thus, the image signal (i.e., Vout) generated by the CCD unit 510 is transmitted to the CDS circuit 505.

The CDS circuit 505 samples the image signal from the CCD unit 510 two times per one reset pulse (i.e., at a timing just after output of the reset pulse and at a timing just before output of the next reset pulse) to obtain a difference between amplitudes of the two values sampled between the reset pulse and the next reset pulse. The difference is outputted from the CDS circuit 505 to the A/D converter 605. According to the above mentioned operation of the CDS circuit 505, 1/f noise and reset pulse noise can be eliminated from the image signal outputted by the CDS circuit 505.

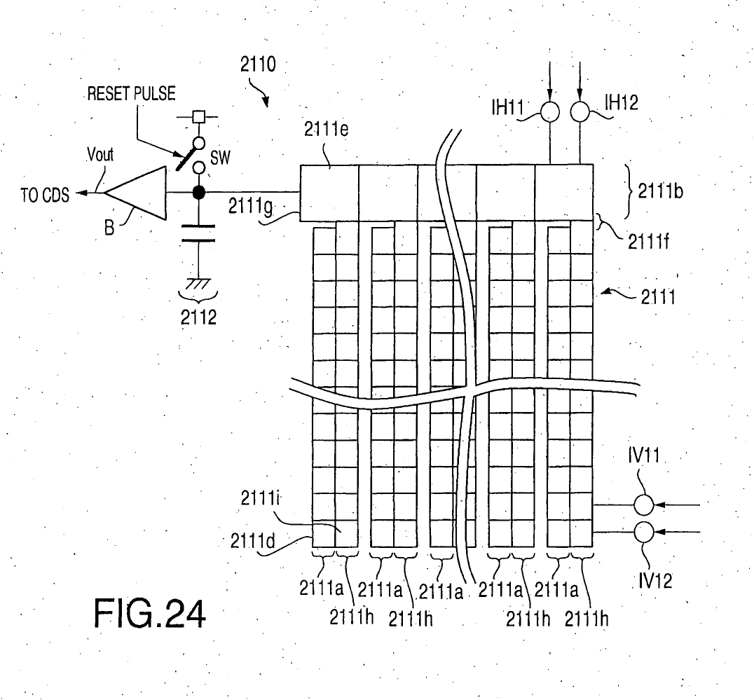

Although in this embodiment the CCD 511 has the frame-transfer architecture, a CCD having an interline architecture may be used in place of the CCD 511. FIG. 24 shows a CCD unit 2110 having the interline architecture. The CCD unit 2110 includes a CCD 2111 having the interline architecture, a Floating Diffusion Amplifier (i.e., a charge detection amplifier) 2112, and a buffer B.

The CCD 2111 includes N columns of light receiving linens 2111a on which the image is formed by the objective lens 501, a horizontal transferring CCD (HCCD) 2111b, vertical transferring CCDs (VCCD) 2111h, horizontal pulse input terminals IH1 and IH2, and vertical pulse input terminals IV1 and IV2. As shown in FIG. 24, each light receiving line 2111a has M cells 2111d aligned vertically. That is, the CCD 2111 has the M by N matrix of cells 2111d for receiving light from the object.

Neighboring to each light receiving line 2111a, one vertical transferring CCD (VCCD) 2111h is located. The VCCD 2111h also includes M cells 2111i aligned vertically. Cells 2111d in each light receiving line 2111a are respectively connected to the cells 2111i in the neighboring VCCD 2111h. Areas of the VCCDs 2111h are covered with optical shielding, and light from the object is not incident on the VCCDS 2111h. Therefore, only the cells 2111d of light receiving lines 2111a receive light from the object.

The HCCD 2111b is located along one side 2111f of the CCD 2111. The HCCD 2111b has N cells 2111e which are aligned in a line horizontally and which respectively correspond to the VCCDs 2111h. One of the cells 2111e located at one end (leftward end in FIG. 24) of the HCCD 2111b is connected to the FDA 2112.

As in the case of the CCD 511 having the frame-transfer architecture, the horizontal pulse input terminals IH1 and IH2, and the vertical pulse input terminals IV1 and IV2 are connected to the driver circuit 504, and receive the timing pulses from the driver circuit 504. Each of the vertical pulse input terminals IV1 and IV2 is connected to each cell 2111d. Each of the horizontal pulse input terminals IH1 and IH2 is connected to each cell 2111e of the HCCD 2111b.

While the image is formed by the objective lens 501 on the cells 2111d, charge is accumulated in each cell 2111d. The amount of charge accumulated in each cell 2111d depends on the accumulation period and a luminance of the optical image. Each time pulses are inputted to the vertical pulse input terminals IV1 and IV2, charge transferred from each cell 2111d to each cell 2111i is transferred vertically to its neighboring cell toward the HCCD 2111b. Also, charges accumulated in cells 2111d arranged along the side 2111f are respectively transferred to the cells 2111e of the HCCD 2111b.

In this example shown in FIG. 24, charge is transferred vertically based on the two-phase transferring technology. However, another technology, such as a single phase transferring technology, three-phase transferring technology, and four-phase transferring technology well known in the art, may be adopted to transfer charge vertically.

Each time a pair of pulses are inputted to the horizontal pulse input terminals IH1 and IH2, charge accumulated in each cell 2111e is transferred horizontally (i.e., leftward in FIG. 24) to its neighboring cell. Also, charge accumulated in one of cells 2111e located nearest to the FDA 2112 is transferred to the FDA 2112. In this example, charge is transferred horizontally based on the two-phase transferring technology. However, another technology, such as a single phase transferring technology, three-phase transferring technology, and four-phase transferring technology well known in the art, may be adopted to transfer charge horizontally.

As described above, the CCD unit 2110 can be controlled in the same way as that of the CCD unit 510. Functions of the FDA 2112 and the buffer B are the same as those shown in FIG. 23, detailed explanation thereof will not be repeated.

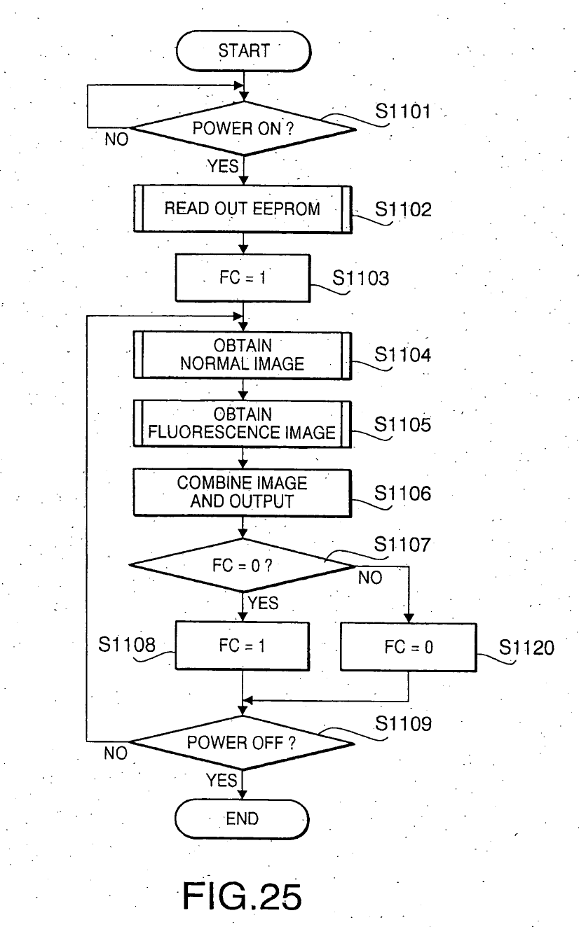

Hereafter, an image processing performed under control of the MPU 601 provided in the endoscope processor 600 will be described. As described below, images for normal observation (i.e., color images) and images for fluorescent observation (i.e., fluorescence images) are obtained in the image processing. FIG. 25 is a flowchart (a main flow) showing the image processing. A program of the image processing is stored, for example, in the embedded ROM and is read out by the MPU 601.

Firstly, MPU 601 determines whether power of the endoscope 500 is ON or OFF (step S1101). When the power of the endoscope 500 is OFF (S1101:NO), the MPU 601 repeats the step S1101. When the power of the endoscope 500 is ON (S1101:YES), control proceeds to step S1102.

In step S1102, the MPU 601 reads out information from the EEPROM 506 and assigns various parameters to variables. In step 1103, 1 is assigned to a variable FC (FC=1), that indicates odd-field or even-field for video signal. Next, in step S1104, the image for the normal observation (i e., the normal image) is obtained. Next, in step S1105, the image for the fluorescent observation (i.e., the fluorescence image) is obtained.

In step S1106, the image for normal observation obtained in step S1104 and the image for the fluorescent observation obtained in step S1105 are combined. Then the combined image is converted to the video signal and is outputted to the monitor 690. The video signal is outputted as an odd-field of an interlace scan signal when FC=1, and is outputted as an even-filed of the interlace scan signal when FC=0.

Next, the MPU 601 determines whether FC is zero or not (S1107). When FC is 0 (S1107:YES), 1 is assigned to FC (S1108). When FC is 1 (S1107:NO), 0 is assigned to FC (S1120). Then, in step S1109, the MPU 601 determines whether the power of the endoscope 500 is OFF or ON.

When the endoscope 500 is ON (S1109:NO), control returns to step S1104 to obtain a next field of the image. When the endoscope 500 is OFF (S1109:YES), the image processing terminates. According to the image processing shown FIG. 25, both of the image for the normal observation and the image for the fluorescent observation can be obtained and are displayed on the monitor 690.

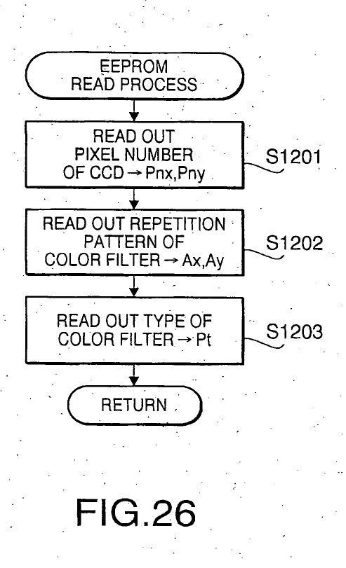

FIG. 26 is a flowchart showing an EEPROM read process performed in step S1102 of FIG. 25.

Firstly, in step S1201, the MPU 601 obtains the number of cells 511d provided in the light receiving unit 511a. More specifically, the MPU 601 obtains the number of cells 511d in one row (i.e., the cells 511d aligned in the horizontal direction) and the number of cells 511d in one column (i.e., the cells 511d aligned in the vertical direction) from the EEPROM 506. The MPU 601 assigns the number of cells 511d in one row to a variable Pnx, and assigns the number of cells 511d in one column to a variable Pny.

As described above, in this embodiment, the cells 511d are aligned in M by N matrix. The number N is assigned to the Pnx and the number M is assigned to the Pny.

On cells 511a of the color CCD 511, a color filter is formed. Examples of the color filter has been described with reference to FIGS. 13-15 of the second embodiment. these examples can also be used in the third embodiment. Therefore, explanations about examples of the color filters will not be repeated. In this embodiment, the color filter shown in FIG. 13 is used.

In step S1202, information concerning a repetition pattern of the filter elements of the color filter is read out from the EEPROM 506. More specifically, the information concerning the repletion pattern includes the number of filter elements arranged in the horizontal direction in one group 91 (see FIG. 13), and the number of filter elements arranged in the vertical direction in one group 91.

In the case of the color filter shown in FIG. 13, the number of filter elements arranged in the horizontal direction in one group 91 is 2, and the number of filter elements arranged in the vertical direction in one group 91 is 2. These obtained numbers (2, 2) are stored in variables (AX, Ay), respectively.

After the information concerning the repetition pattern of the filter elements is obtained in step S1202, control proceeds to step S1203. In step S1203, the MPU 601 obtains an ID number indicating the type of the color filter array pattern of the color filter in the color CCD 510 from the EEPROM 506.

For example, the ID number of the type of the color filter array pattern is zero when the color filter shown in FIG. 13 is used, the ID number of the type of the color filter array pattern is 1 when the color filter shown in FIG. 14 is used, and the ID number of the type of the color filter array pattern is 2 when the color filter shown in FIG. 15 is used. The ID number of the type of the color filter array pattern is stored in a variable Pt.

After step S1203 is processed, control returns to step 1103 of the main flow (FIG. 25).

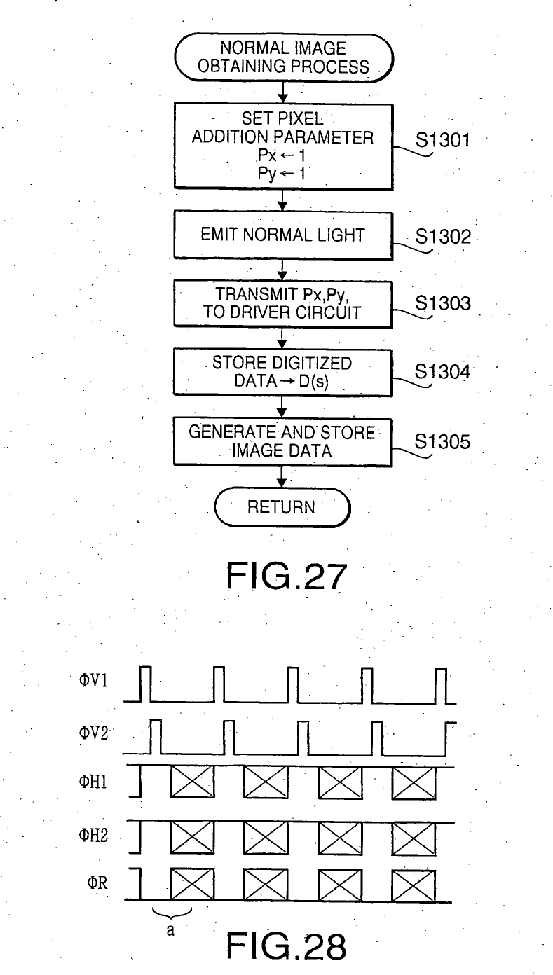

FIG. 27 is a flowchart showing a normal image obtaining process performed in step S1104 of the main flow (FIG. 25). Firstly, in step S1301, 1 is assigned to each of the variables Px and Py. Next, in step S1302, normal light (i.e., white light) is emitted by the light source 602.

In step S1303, the values of Px and Py are sent to the driver circuit 504. The driver circuit 504 uses the Px and Py to generate timing signals to be inputted to the CCD unit 510 and the CDS circuit 505. By performing the step S1303, the image signal corresponding to one field is outputted by the CDS circuit 505 to the A/D converter 605.

In step S1304, the MPU 601 controls the A/D converter 605 and the DSP 603 to store the digital signal digitized. Then, in step S1305, the MPU 601 controls the A/D converter 605 and the DSP 603 to convert the digitized image signal to image data and to store the image data in the memory 604. Various parameters obtained in step S1102 in the main flow is used in the conversion step S1305. Hereafter, the digitized image signal corresponding to one field of one frame is represented by pixel data P(x,y) having resolution of Pnx×Pny where 0≦x<Pnx, 0≦y<Pny.

Similarly to the conversion calculation in the second embodiment, the DSP 603 converts the pixel data P(x,y) to red, green and blue component image data of R(x,y), G(x,y) and B(x,y) using the above mentioned equations (1)-(3), according to the type of the color filter. R(x,y) represents the red component intensity value of a pixel having coordinates (x,y). G(x,y) represents the green component intensity value of a pixel having coordinates (x,y). B(x,y) represents the blue component intensity value of a pixel having coordinates (x,y).

The digital image data of one pixel when the color filter shown in FIG. 13, FIG. 14 or FIG. 15 is used is thus obtained from charges accumulated in four or three cells 511d according to the above equations (1)-(3).

After the above mentioned calculation is performed in step S1305 of FIG. 27, the normal image obtaining process terminates, and control returns to step S1105 of the main flow (FIG. 25).

Next, operation of the driver circuit 504, the CDS circuit 505 and the CCD unit 510 when the MPU 601 sends the Px and Py to the driver circuit 504 in step S1303 of FIG. 27 will be explained. FIG. 28 is a timing chart showing the timing signals outputted by the driver circuit 504 when the driver circuit 504 receives the Px and Py from the MPU 601. That is, the timing chart shows operation when the image for the normal image is obtained.

As shown in FIG. 28, the driver circuit 504 outputs a φV1 signal, a φV2 signal, a φH1 signal, a φH2 signal and a φR signal (i.e., the reset pulse). The φV1 signal and the φV2 signal are respectively inputted to the vertical pulse input terminals IV1 and IV2 of the CCD unit 510. The φH1 signal and the φH2 signal are respectively inputted to the horizontal pulse input terminals IH1 and IH2 of the CCD unit 510. The φR signal is inputted to the switch SW of the FDA 512.

As shown in FIG. 28, when a pair of successive one φV1 pulse and one φV2 pulse is inputted to the input terminals IV1 and IV2, charge accumulated in each cell 511d is transferred vertically (i.e., upward in FIG. 23) to its neighboring cell 511d. Then, by respectively inputting the φH1 pulses and φH2 pulses to the input terminals IH1 and IH2, charge accumulated in each cell 511e in the HCCD 511b is outputted from the HCCD 511b to the FDA 512. Further, by inputting the φR pulses to the CCD unit 510, the image signal is sent from the unit CCD 510 to the CDS circuit 505.

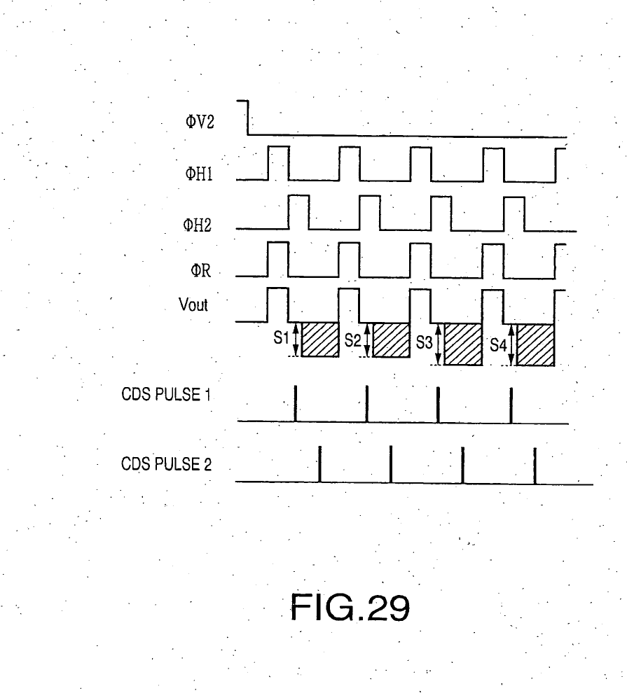

FIG. 29 is a timing chart showing the timing within a timing period “a” of FIG. 28 in detail. As shown in FIG. 18, the φR pulses are inputted to the CCD unit 510 in synchronization with the φH1 pulses. Also, in FIG. 29, CDS pulses 1 and 2 which are sent from the driver circuit 504 to the CDS circuit 505 are indicated.

The CDS circuit 505 samples the image signal from the CCD unit 510 when the CDS pulse 1 is inputted thereto and when the CDS pulse 2 is inputted thereto. Then the CDS circuit 505 obtains a difference between amplitude of the image signal when the CDS pulse 1 is inputted and amplitude of the image signal when the CDS pulse 2 is inputted.

More specifically, the CDS pulse 1 is asserted just after the falling edge of the R pulse, and is asserted just before the rising edge of the next φR pulse. Synchronizing with the falling edge of the φH2 pulse charge accumulated in each cell 511e of the HCCD 511b is transferred to its neighboring cell. The FDA 512 accumulates charge from the cell 511e of the HCCD 511b, and outputs the voltage level corresponding to the charge accumulated therein. Consequently, a signal Vout (i.e., the image signal) corresponding to the charge accumulated in the FDA 512 is outputted from the buffer B to the CDS circuit 505.

According to the above mentioned operation of the CCD unit 510 and the CDS circuit 505, the difference between amplitude of the image signal sampled when the CDS pulse 1 is asserted and amplitude of the image signal when the CDS pulse 2 is asserted appropriately indicates the voltage level corresponding to charge accumulated in the FDA 512.

That is, the CDS circuit 505 outputs voltage levels respectively corresponding to voltage levels of S1, S2, S3 and S4 shown in FIG. 29. As shown in FIG. 29, one φR pulse is asserted per one φH1 pulse. Therefore, each of voltage levels S1, S2, S3 and S4 corresponds to charge accumulated in one cell 511e.

As describe above, Charge accumulated in one cell 511d is transferred to each cell 511e of the HCCD 511b when the pair of signals φV1 and φV2 is asserted. Therefore, each of the voltage levels of S1, S2, S3 and S4 shown in FIG. 29 corresponds to one cell 511d of the light receiving unit 311a.

Consequently, the CDS circuit 505 sequentially outputs voltage levels respectively corresponding to charges in the cells 511d.

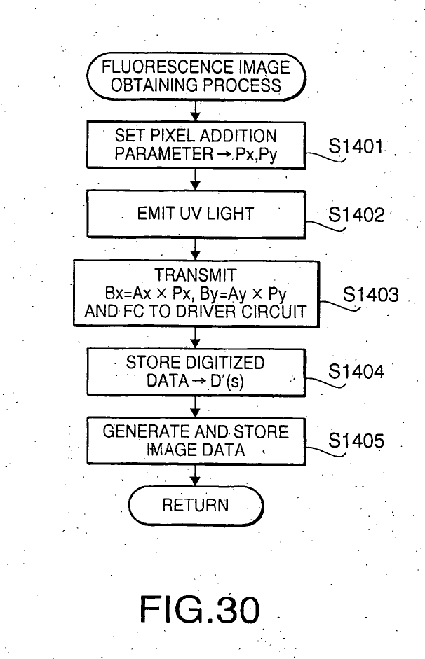

FIG. 30 is a flowchart showing in detail a process of obtaining the fluorescence image (hereafter, referred to as a fluorescence image obtaining process) performed in step S1105 of the main flow (FIG. 25).

Firstly, in step S1401, pixel addition parameters are assigned to Px and Py. The pixel addition parameters represent the number of pixels of the normal image to be added together for generating one pixel of the fluorescence image.

For example, if Px=2 and Py=3, intensities of two cell groups (e.g., two color groups 91 in FIG. 13) aligned in the horizontal direction and intensities of three cell groups aligned in the vertical direction are added together, and the sum is used as an intensity of one pixel of the fluorescence image. That is, 2 by 3 pixel groups are used to generate one pixel of the fluorescence image. The pixel addition parameters may be set by a user to the endoscope processor 600 for example by user operation. In this embodiment, Px and Py are even numbers.

As described above, one color group (e.g., the color group 91 in FIG. 13) of the color filter has different types of color filter elements. It is noted that the different types of color filter elements have different transmissivity. Therefore, in order to match a gray scale of the fluorescence image to a real image of the object, the number of cells 511d used for generation of one pixel of the fluorescence image has to be integral multiples of the number of cells 511d included in one color group.

That is, the number of cells 511d in the horizontal direction used to generate one pixel of the fluorescence image has to be integral multiples of the number of cells 511d in the horizontal direction used to generate one pixel of the normal image. Also, the number of cells 511d in the vertical direction used to generate one pixel of the fluorescence image has to be integral multiples of the number of cells 511d in the vertical direction used to generate one pixel of the normal image.

In step S1402, the MPU 601 controls the light source 602 to emit UV light (i.e., the excitation light). Next, in step S1403, the product of Ax and Px is assigned to a variable Bx, and the product of Ay and Py is assigned to a variable By. Then, the MPU 601 sends the variables Bx and By to the driver circuit 504. The driver circuit 504 generates the timing signals based on the variables Bx and By. By performing the step S1403, the image signal corresponding to one field is outputted by the CDS circuit 505.

Next, in step S1404, the MPU 601 controls the A/D converter 605 and the DSP 603 to store digitized image signal D'(s) corresponding to one field. Then, in step S1405, the MPU 601 controls the A/D converter 605 and the DSP 603 to convert the digitized image signal from the CDS circuit 505 to image data. The image data is then stored In the memory 604. The various parameters obtained by the MPU 601 in step S1102 of the main flow are used in the conversion process in step S1405.

Hereafter, a frame of the digitized signal generated by the A/D converter 605 is represented by P′(x,y) having the pixel number of Pnx/Bx×Pny/By (where 0≦x<Pnx/Bx, 0≦y<Pny/By). The DSP 603 converts the P′(x,y) to image data F(x,y). The F(x,y) represents an intensity value of one pixel data of the fluorescence image having the horizontal coordinate x and the vertical coordinate y.

The DSP 603 determines the F(x,y) according to the following equations.

0≦x<Pnx/Bx−1

0≦y<Pny/By−1

F(x,y)=P′(x,y)

After the image data is obtained instep S1405, the fluorescence image obtaining process terminates. Then, control returns to step S1106 of the main flow.

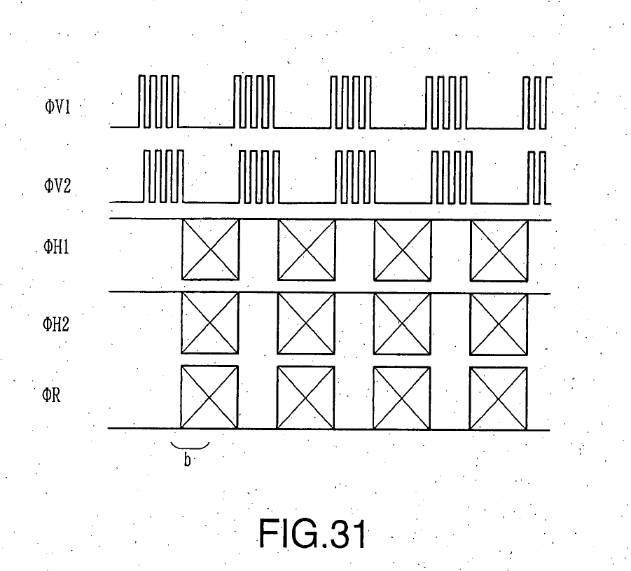

The operation of the driver circuit 504, the CCD unit 510 and the CDS circuit 505 when the Bx, By and FC are transmitted from the MPU 601 to the driver circuit 504 will be described below. FIG. 31 is a timing chart showing the timing signals outputted by the driver circuit 505 when the Bx, By and FC (FC=1) are transmitted from the MPU 601 to the driver circuit 505.

As show in FIG. 31, the driver circuit 504 outputs successive “By” φV1 pulses (i.e., 4 pulses in the example shown in FIG. 31) and successive “By” φV2 pulses while the φH1 and φH2 are high in order to transfer charge accumulated in each cell 511d vertically by “By” steps (i.e., 4 steps in the example shown in FIG. 31) toward the HCCD 511b. After the four successive φV1 pulses and the four successive φV2 pulses are asserted, φH1 pulses and φH2 pulses are asserted successively to transfer charges accumulated in cells 511e to the FDA 512, and φR pulses are also asserted to transmit the image signal from the CCD unit 510 to the CDS circuit 505.

By inputting the φV1 pulses and φV2 pulses as described above, charges of By cells 511d (i.e., four cells 511d in the example shown in FIG. 31) aligned in the vertical direction are added together in each cell 511e of the HCCD 511b.

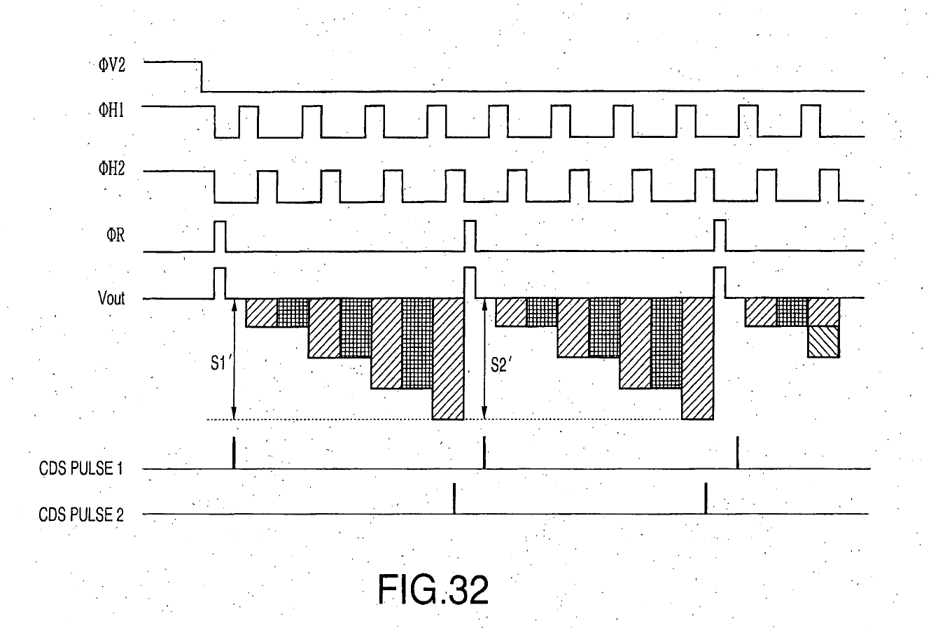

FIG. 32 is a timing chart showing in detail the timing of the φH1, φH2, φR, Vout (i.e., the image signal), the CDS pulse 1 and the CDS pulse 2 during a time period “b” indicated in FIG. 31. As shown in FIG. 32, the φR pulse is asserted one time while φH1 pulses are asserted Bx times (i.e., four times in an example of FIG. 32). Each φR pulse is asserted in synchronization with its corresponding pulse φH1.

When the φH2 pulse falls, charge accumulated in each cell 511e of the HCCD 511b is transferred leftward to its neighboring cell 511e. The φH2 pulse is asserted Bx times (i.e., four times in the example shown in FIG. 32) during an interval between two successive φR pulses. The FDA 512 starts to accumulate charge from the HCCD 511b after the φR pulse falls. Therefore, in the interval between the two φR pluses, charges of Bx cells 511e (i.e., charges of four cells 511e in the example shown in FIG. 32) are added together into the FDA 512, and are converted to a voltage level by the FDA 512.

The CDS pulse 1 is asserted just after the falling edge of the φR pulse, while the CDS pulse 2 is asserted just before the rising edge of the next φR pulse. Consequently, the CDS circuit 505 outputs voltage levels respectively corresponding to amplitudes S1′ and S2′ indicated in FIG. 32.

It is understood from the above explanation that each of the amplitude S1′ and amplitude S2′ corresponds to charges accumulated in successive Bx cells 511e in the HCCD 511b. In addition, as described above, charges of By (i.e., four) cells 511d aligned in the vertical direction are added together in each cell 511e of the HCCD 511b. Therefore, each of the amplitude S1′ and amplitude S2′ corresponds to the sum of charges accumulated in cells 311d arranged in By×Bx matrix.

As described above, Bx is the product of Ax and Px, and By is the product of Ay and Py. Therefore, by inputting the above mentioned timing pulses to the CCD unit 510 and the CDS circuit 505, the CDS circuit 505 outputs sequentially voltages levels, each of which corresponds to integral multiple of one pixel of the normal image.

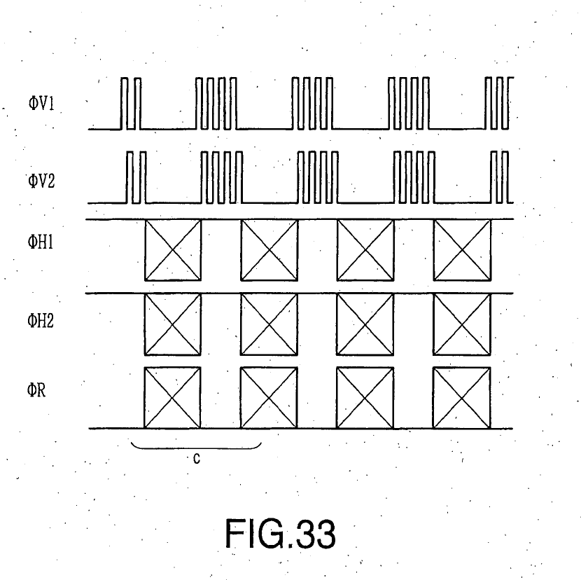

FIG. 33 is a timing chart showing the timing signals outputted by the driver circuit 505 when the Bx, By and FC (FC=0) are transmitted from the MPU 601 to the driver circuit 505 in step S1403 of FIG. 30.

As show in FIG. 33, the driver circuit 504 outputs successive “By,” φV1 pulses (i.e. 4 pulses in the example shown in FIG. 33) and successive “By” φV2 pulses while the φH1 and φH2 are high in order to transfer charge accumulated in each cell 511d vertically by “By” steps (i.e., 4 steps in the example shown in FIG. 33) toward the HCCD 511b. After the four successive φV1 pulses and the four successive φV2 pulses are asserted, φH1 pulses and φH2 pulses are asserted successively to transfer charges accumulated in cells 511e to the FDA 512, and φR pulses are also asserted to transmit the image signal from the CCD unit 510 to the CDS circuit 505.

By inputting the φV1 pulses and φV2 pulses as described above, charges of By cells 511d (i.e., four cells 511d in the example shown in FIG. 33) aligned in the vertical direction are added together in each cell 511e of the HCCD 511b.

In contrast to the case of FC=1, at the top of the field, only By/2 (i.e., two) successive φV1 pulses and successive By/2 (i.e., two) φV2 pulses are asserted when FC is 0.

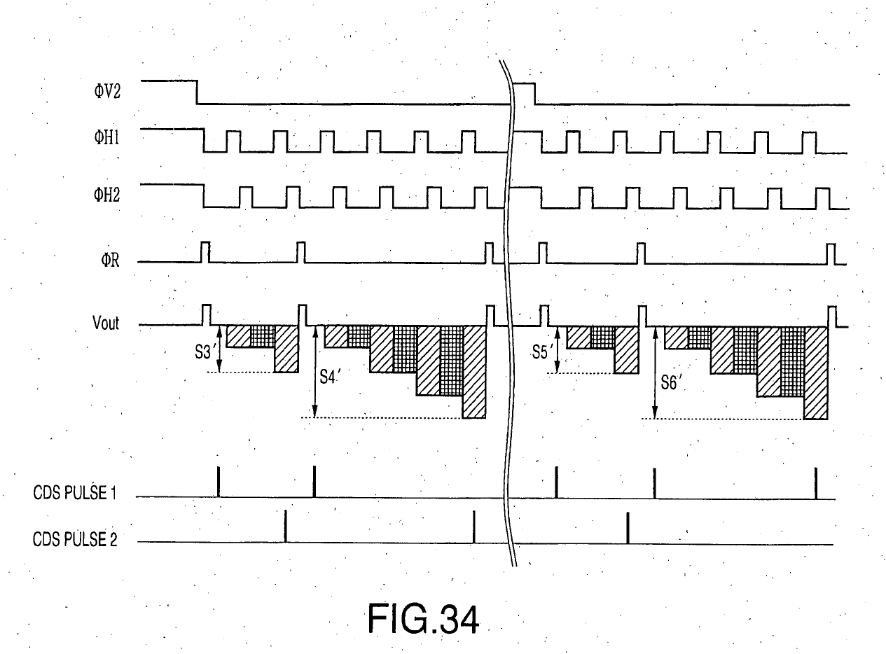

FIG. 34 is a timing chart showing in detail the timing of the φH1, φH2, φR, Vout (i.e., the image signal), the CDS pulse 1 and the CDS pulse 2 during a time period “c” indicated in FIG. 33. As shown in FIG. 34, the φR pulse is asserted one time while φH1 pulses are asserted Bx times (i.e., four times in an example of FIG. 34). Each φR pulse is asserted in synchronization with its corresponding pulse φH1.

However, in contrast to the case when FC is 1, the φR pulse is asserted one time while φH1 pulses are asserted Bx/2 times (i.e., two times in an example of FIG. 34).

When the φH2 pulse falls, charge accumulated in each cell 511e of the HCCD 511b is transferred leftward to its neighboring cell 511e. The φH2 pulse is asserted Bx times (i.e., four times in the example shown in FIG. 34) during an interval between two successive φR pulses. The FDA 512 starts to accumulate charge from the HCCD 511b after the φR pulse falls. Therefore, in the interval between the two φR pluses, charges of Bx cells 511e (i.e., charges of four cells 511e in the example shown in FIG. 34) are added together into the FDA 512, and are converted to a voltage level by the FDA 512.

The CDS pulse 1 is asserted just after the falling edge of the φR pulse, while the CDS pulse 2 is asserted just before the rising edge of the next φR pulse. Consequently, the CDS circuit 505 outputs voltage levels respectively, corresponding to amplitudes S3′, S4′, S5′ and S6′ indicated in FIG. 34.

The amplitude S3′ corresponds to a first output of the top line of the filed, the amplitude S4′ corresponds to a second output of the top line of the field, the amplitude S5′ corresponds to a first output of the second line of the field, and the amplitude S6′ corresponds to a second output of the second line of the field.

It is understood from the above explanation that each of the amplitude S3′ and amplitude S5′ corresponds to charges accumulated in successive Bx/2 cells 511e in the HCCD 511b, and that each of the amplitude S4′ and amplitude S6′ corresponds to charges accumulated in successive Bx cells 511e in the HCCD 511b. In addition, as described above, charges of By (i.e., four) cells 511d aligned in the vertical direction are added together in each cell 511e of the HCCD 511b except for an, exception in which By/2 cells 511d aligned in the vertical direction are added together in each cell 511e with regard to the top line of the field.

Therefore, the amplitude S3′ corresponds to the sum of charges accumulated in Bx/2×By/2 cells 311d, the amplitude S4′ corresponds to the sum of charges accumulated in Bx×By/2 cells 311d, the amplitude S5′ corresponds to the sum of charges accumulated in Bx/2×By cells 311d, and the amplitude S6′ corresponds to the sum of charges accumulated in Bx×By cells 311d.

As described above, Bx is the product of Ax and Px, and By is the product of Ay and Py. Therefore, by inputting the above mentioned timing pulses to the CCD unit 510 and the CDS circuit 505, the CDS circuit 505 outputs sequentially voltages levels, each of which corresponds to integral multiple of one pixel of the normal image.

According to the embodiment of the invention, the image formed on the light receiving unit 511 can be amplified Bx×By times when the interlace scan image is obtained.

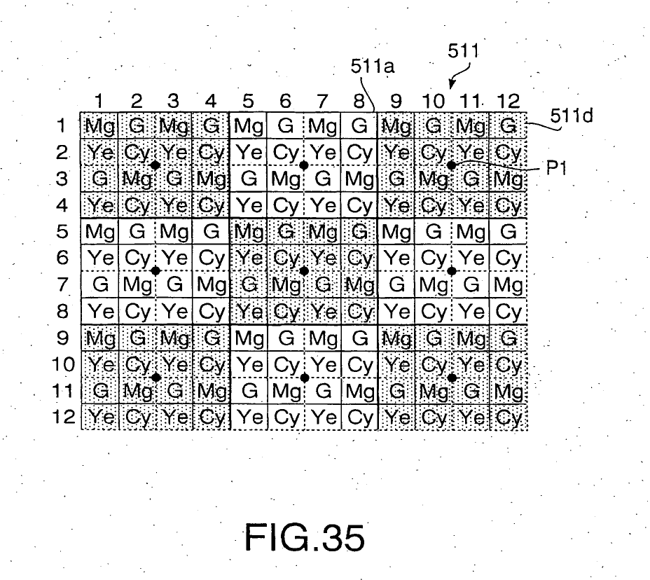

FIG. 35 shows the upper left portion of the CCD 511 on which the color filter shown in FIG. 13 is formed, schematically illustrating cells 511d which are used to generate one field of the fluorescence image when the parameters are FC=1, Ax=2, Ay=2, Px=2 and Py=2. In FIG. 35, the cells 511d indicated by hatching patterns (i.e., cells 511d surrounded by a heavy line box) are used to generate one field of the fluorescence image when the parameters are FC=1, Ax=2, Ay=2, Px=2 and Py=2. Each cell group surrounded by the heavy line box, which is represented by a dot P1, corresponds to one pixel of the fluorescence image.

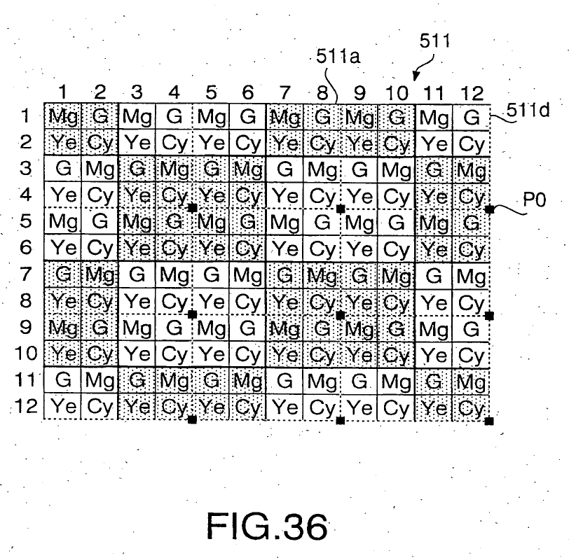

FIG. 36 shows the upper left portion of the CCD 511 on which the color filter shown in FIG. 13 is formed, schematically illustrating cells 511d which are used to generate one field of the fluorescence image when the parameters are FC=0, Ax=2, Ay=2, Px=2 and Py=2. In FIG. 36, the cells 511d indicated by hatching patterns (i.e., cells 511d surrounded by a heavy line box) are used to generate one field of the fluorescence image when the parameters are FC=1, Ax=2, Ay−2, Px=2 and Py=2. Each cell group surrounded by the heavy line box, which is represented by a dot P0, corresponds to one pixel of the fluorescence image.

As can be seen from FIGS. 35 and 36, a position of each cell group P0 in the even field of the image is shifted by half pixel size in the horizontal direction and in the vertical direction in comparison with a position of each cell group P1 in the odd field of the image. That is, a position on the cells 511d when the even field is obtained is different from position on the cell 511d when the odd field is obtained even if the coordinates on the fluorescence image are the same.

The resolution of each field of the interlace scan image becomes Pxn/Bx pixels in the horizontal direction and Pyn/By/2 in the vertical direction.

Although the present invention has been described in considerable detail with reference to certain preferred embodiments thereof, other embodiments are possible.

The present disclosure relates to the subject matters contained in Japanese Patent Applications No. P2003-094996, filed on Mar. 31, 2003, No. P2003-094997, filed on Mar. 31, 2003, and No. P2003-094998, filed on Mar. 31, 2003, which are expressly incorporated herein by reference in their entireties.

What is claimed is:

1. An endoscope system, comprising:
an endoscope that has an image pick-up device having a plurality of cells for accumulating charge in response to an image formed on the plurality of cells;
a light source that is capable of emitting excitation light; and
a controlling system that controls said image pick-up device so that charges accumulated in two or more of the plurality of cells are added together when the excitation light is used.

2. The endoscope system according to claim 1, wherein said image pick-up device includes a charge coupled device.

3. The endoscope system according to claim 2, wherein said charge coupled device has a frame-transfer architecture.

4. The endoscope system according to claim 2, wherein said charge coupled device has an interline architecture.

5. The endoscope system according to claim 2,
wherein said controlling system includes:
a driver circuit that outputs timing signals to said charge coupled device, said charge coupled device outputting charge accumulated in said plurality of cells in accordance with the timing signals;
a detection amplifier that accumulates charge outputted by said charge coupled device and outputs voltage levels in accordance with the charge accumulated therein; and
a correlated double sampling circuit that samples the voltage levels outputted by said detection amplifier and outputs a sampled signal.

6. The endoscope system according to claim 5,
wherein the plurality of cells in said charge coupled device are arranged in a matrix,
wherein said charge coupled device further includes a horizontal transferring charge coupled device connected to the plurality of cells in the charge coupled device, the horizontal transferring charge coupled device outputting the charge to said detection amplifier.

7. The endoscope system according to claim 6,
wherein charge accumulated in each of the plurality of cells arranged in the matrix is transferred vertically a plurality of times toward the horizontal transferring charge coupled device so that charge of at least two cells aligned vertically in the plurality of cells is accumulated in each cell of the horizontal transferring charge coupled device.

8. The endoscope system according to claim 6,
wherein charge accumulated in each of cells in the horizontal transferring charge coupled device is transferred horizontally a plurality of times so that charge of at least two cells in the horizontal transferring charge coupled device is accumulated in said detection amplifier.

9. The endoscope system according to claim 7,
wherein said driving circuit transmits reset pulses to said detection amplifier to discharge the charge accumulated in said detection amplifier, and transmits sampling signals to said, correlated double sampling circuit to sample the voltage levels outputted by said detection amplifier,
wherein said driving circuit controls timing of the reset pulses and the sampling signals so that charge of the at least two cells aligned vertically in the plurality of cells is accumulated in each cell of the horizontal transferring charge coupled device.

10. The endoscope system according to claim 1, further comprising an image processing system that generates an image for fluorescent observation so that an intensity of one pixel of the image corresponds to the added charges of the two or more of the plurality of cells.

11. The endoscope system according to claim 1,
wherein said image pick-up device includes a color CCD having color filter covering the plurality of cells,
wherein said controlling system controls said color CCD so that a number of cells whose charges are added together becomes an integral multiple of a number of filter elements of the color filter to be used for generating a pixel of an image for fluorescent observation.

12. The endoscope system according to claim 11, further comprising a memory that stores information concerning a repetition pattern of the filter elements
wherein said controlling system obtains the number of filter elements to be used for generating the pixel of the image for fluorescent observation based on the information stored in said memory.

13. The endoscope system according to claim 1,
wherein said image pick-up device includes a color CCD having color filter covering the plurality of cells,
wherein said controlling system controls said color CCD, so that a number of cells whose charges are added together becomes an integral multiple of a number of filter elements of the color filter to be used for generating a pixel of an image for fluorescent observation,
wherein said control system controls said color CCD so that a combination of the filter elements, to which the two or more cells added together correspond, is changed between an odd field and an even field.

14. The endoscope system according to claim 13, further comprising an image processing system that generates an interlace scan image signal so that an intensity of one pixel of the interlace scan image signal corresponds to the added charges of the two or more of the plurality of cells.
